(19) United States
(12) Patent Application Publication
(10) Pub. No.: US 2024/0246842 A1
(43) Pub. Date: Jul. 25, 2024

(54) Immobilized Microbes for Water Treatment

(71) Applicant: Novozymes A/S, Bagsvaerd (DK)

(72) Inventors: Cedric Hobel, Lyngby (DK); Alexander Findeisen, Bagsvaerd (DK); Sebastian Reinhold Soerensen, Hilleroed (DK); Pavle Andric, Copenhagen (DK); Nicholas Bagger Gurieff, Queensland (AU)

(73) Assignee: Novozymes A/S, Bagsvaerd (DK)

(21) Appl. No.: 18/554,192

(22) PCT Filed: Apr. 26, 2022
(86) PCT No.: PCT/EP2022/061109

§ 371 Date: Oct. 5, 2023

(30) Foreign Application Priority Data
Apr. 26, 2021 (EP) 21170527.2

(51) Int. Cl. C02F 3/10; C02F 3/34; C02F 101/16; C12N 1/20; C12N 11/084

(52) U.S. Cl. CPC C02F 3/108; C02F 3/341; C12N 1/20; C12N 11/084; C02F 2101/163; C02F 2303/02

(57) ABSTRACT

A polymer support of immobilized microorganisms has been shown to be effective in reducing total nitrogen content, removing ammonia, nitrates and nitrites and suitable for the cleaning of wastewater and for the preparation and cleaning of drinking water.

### FIELD OF THE INVENTION

The present invention relates to microorganisms immobilized on a polymer support for nitrogen removal from drinking water or wastewater.

### BACKGROUND OF THE INVENTION

The presence of various nitrogen species in both drinking water and wastewater can be problematic and even toxic for humans, wildlife and the environment. Therefore, it is important that treatment processes are in place to protect both human health and the environment.

Modern wastewater treatment plants have been designed to meet effluent standards for nitrogen discharge. However, as discharge regulations are tightened, enhanced nitrogen removal processes are required to supplement the normal nitrogen removal processes already in place. These enhanced processes can involve the nitrification of ammonium to nitrate or the dentification of nitrate and nitrate to nitrogen, or a combination of both processes. Generally, biological sand filters or other biological filters are used for this purpose as they can ensure that the nitrogen is treated as needed, but also limit the amount of biomass (bacteria) that is discharged in the effluent as a by-product of the enhanced nitrogen treatment.

The shortcomings of existing nitrogen treatment processes include but are not limited to: required cleaning cycles in biological filters reducing operational time, the relatively low concentration of active microorganisms in biological filters, cost of operation and the production of a brine by ion exchange membranes that then requires further treatment.

The goal of wastewater treatment plants according to the invention is to treat the incoming wastewater to a standard where it can then be responsibly discharged to the environment. An important aspect of modern wastewater treatment is the removal of nutrients that can cause damage to the environment the treated wastewater is discharged to, including nitrogen.

Domestic sewage typically contains 20 to 40 mg/L (ppm) of ammonia nitrogen (NH₄—N). Organic matter containing nitrogen, e.g., protein and nucleic acid, also biodegrades to release ammonia. Releasing this ammonia into receiving streams has a direct toxic effect on fish and other animals and, in addition, causes significant oxygen depletion as illustrated in the following equation.

The presence of various nitrogen species in both drinking water and wastewater can be problematic and even toxic for humans, wildlife and the environment. Therefore, it is important that treatment processes are in place to protect both human health and the environment.

With regards to wastewater treatment, the presence of nitrogen in any form in treated effluent can be harmful to the environment the treated wastewater is being discharged to. Ammonium is directly toxic to fish and other aquatic lifeforms, while excessive levels of nitrate and nitrite can lead to eutrophication in water courses, leading to fish die offs, odours and other environmental issues.

In general, nitrogen treatment in water by biological treatment processes involve the processes are known as nitrification (oxidation of ammonium (NH₄⁺) to nitrite (NO₂⁻) and then nitrate (NO₃⁻) by a community of nitrifying microorganisms) and denitrification (reduction of nitrate and/or nitrite to elemental nitrogen (N₂) by a community of denitrifying microorganisms), thereby removing the nitrogen from the water.

In drinking water, the presence of ammonium (NH₄⁺) will lead to nitrification in the distribution network that can lead to aesthetic issues (taste and odour), corrosion, alkalinity consumption and decreased pH. The presence of ammonium can also increase chlorine demand, which then increases the presence of disinfection by-products and increases the potential for unwanted growth in distribution systems. In areas where drinking water sources are anoxic groundwater resources, the presence of nitrate (NO₃⁻) can be an issue. Nitrate can impact how blood transports oxygen, especially in babies, leading to “blue baby syndrome”.

There is a growing concern with increasing concentrations of nitrate in drinking water resources around the world. While some treatments are effective, these create another problem in that they produce a by-product (brine) that is difficult to treat and expensive to dispose of. Therefore, an efficient and effective biological nitrate removal process is industrially, commercially and environmentally advantageous as it would increase the water recovery rate, would not produce a brine and would directly remove the nitrate from the water, rather than simply up-concentrating it in a brine and displacing the nitrate treatment problem to another problem.

Modern drinking water and wastewater treatment plants have been designed to meet standards for nitrogen concentrations. However, as regulations are tightened, enhanced nitrogen removal processes are required to supplement the normal nitrogen removal processes already in place. These enhanced processes can involve the nitrification of ammonium to nitrate or the dentification of nitrate and nitrate to nitrogen, or a combination of both processes. A range of treatment technologies can be applied, with the technology of choice based on the application, the level of treatment required and standards that are required to be met. For drinking water applications, commonly applied technologies include, but are not limited to, ion exchange resins, reverse osmosis membranes and biological sand filters. For wastewater applications, commonly applied technologies include, but are not limited to, activated sludge, integrated fixed film activated sludge (IFAS), moving bed bio-reactors (MBBR), membrane bio-reactors (MBR) and biological sand filters.

The shortcomings of the existing nitrogen treatment processes should be well understood to one skilled in the art, and include but are not limited to: the production of biomass in the process that then requires further treatment and disposal, the required cleaning cycles in biological filters reducing operational time and producing a waste stream, costs of operation and the production of a brine by ion exchange resins and reverse osmosis membranes that then requires further treatment. However, these processes are commonly used as they provide the treatment capacity required and have been applied widely for many years.

To date, no immobilized microbial technology has been successfully implemented. There is a need for effective and environmentally sound water treatment technologies.

### SUMMARY OF THE INVENTION

The invention is directed to a polymer support for immobilizing microorganisms wherein the polymer support is a polymer hydrogel, said hydrogel comprising polyvinyl alcohol polymeric chains cross linked with glutaraldehyde; and water or an aqueous solution.

The invention provides a polymer support of immobilized microorganisms wherein the polymer support comprises said microorganisms immobilized within a polymer hydrogel; wherein the polymer hydrogel comprises polyvinyl alcohol polymeric chains cross linked with glutaraldehyde; and water or an aqueous solution.

A further aspect is directed to a method of treating water, such as any of drinking water, municipal wastewater or industrial wastewater, such as any of drinking water or municipal wastewater comprising mixing said water a polymer support of immobilized microorganisms, wherein the polymer support comprises said microorganisms immobilized within a polymer hydrogel; wherein the polymer hydrogel comprises polyvinyl alcohol polymeric chains cross linked with glutaraldehyde; and water or an aqueous solution.

A further aspect is directed to a method of reducing the total nitrogen (TN) content in wastewater or in drinking water comprising adding to said water a polymer support of immobilized microorganisms, wherein the polymer support comprises said microorganisms immobilized within a polymer hydrogel; wherein the polymer hydrogel comprises polyvinyl alcohol polymeric chains cross linked with glutaraldehyde; and water or an aqueous solution.

A further aspect is directed to a method of reducing the amount of ammonia in wastewater comprising adding to the wastewater a polymer support of immobilized microorganisms, wherein the polymer support comprises said microorganisms immobilized within a polymer hydrogel; wherein the polymer hydrogel comprises polyvinyl alcohol polymeric chains cross linked with glutaraldehyde; and water or an aqueous solution, typically wherein the microorganism is selected from Nitrosomonas eutropha, Nitrobacter winogradskyi and combinations thereof.

A further aspect is directed to a method of denitrifying water comprising adding to the wastewater a polymer support of immobilized microorganisms, wherein the polymer support comprises said microorganisms immobilized within a polymer hydrogel; wherein the polymer hydrogel comprises polyvinyl alcohol polymeric chains cross linked with glutaraldehyde; and water or an aqueous solution, typically wherein the microorganism is selected from the group consisting of Pseudomonas lini, Paracoccus pantotrophus, Paracoccus versutus and combinations thereof.

A further aspect is directed to a method of reducing the odour of water comprising the use of a polymer support of immobilized microorganisms, wherein the polymer support comprises said microorganisms immobilized within a polymer hydrogel; wherein the polymer hydrogel comprises polyvinyl alcohol polymeric chains cross linked with glutaraldehyde; and water or an aqueous solution.

A further aspect is directed to a method of preparing a polymer support of immobilized microorganisms, said method comprising
1a. Combining in solution a source of alginate, polyvinyl alcohol, and microorganisms into a mixture;
1b. Adding said mixture to a Ca₂⁺-containing solution forming a heterogenous solution comprising a gelate structure;
2a. Adding said gelate structure to a cross-linking solution, said cross-linking solution comprising glutaraldehyde;
2b. Mixing said gelate structure in said cross-linking solution so as to form a polymer support of immobilized microorganisms.

### BRIEF DESCRIPTION OF DRAWINGS

FIG. 1 illustrates a summary of the nitrogen cycle.

FIG. 2: illustrates an embodiment of the method of preparing the polymer support of immobilized microorganisms.

FIG. 3 illustrates the cross-linking of polyvinyl alcohol by glutaraldehyde in sulfuric acid.

FIG. 4 illustrates in FIG. 4A the embodiment of a macropore in the center of the polymer support and in FIG. 4B micropores throughout the volume of the polymer support

FIG. 5 illustrates, as described in Example 5, the TN concentrations in the influent (feed) and effluent for reactors R37 (blank biobeads) and R38 (seeded biobeads) over a period of 129 days of continuous operation

FIG. 6 illustrates, as described in Example 5, the specific TN loading rate and removal activity for the biobeads in reactors R37 and R38 over 129 days of continuous operation

FIG. 7 shows, as described in Example 5, Nitrite production as a proportion of the nitrate removed in each reactor over a period of 129 days of continuous operation

FIG. 8 illustrates, as described in Example 7, the relative abundance of all Eubacteria and of Paracoccus in both the blank biobeads in R37 and the seeded biobeads in R38 after 122 days of continuous operation based on FISH analysis

FIG. 9 illustrates, as described in Example 8, TN concentrations in the influent (feed) and effluent for reactors R38 and R45 over a period of 25 days of continuous operation;

FIG. 10 illustrates, as described in Example 8, the specific TN removal activity for the biobeads in reactors R38 and R45 over 25 days of continuous operation

FIG. 11 illustrates as described in Example 8, the Nitrite production as a proportion of the nitrate removed in each reactor over a period of 25 days of continuous operation

FIG. 12 illustrates, as shown in Example 9, the Specific NH₄ loading rate and removal activity for the biobeads in reactors M06 and M07 over 21 days of continuous operation

FIG. 13 illustrates, as shown in Example 9, the NH₄ concentrations in the influent (feed) and effluent for reactors M06 (blank biobeads) and M07 (seeded biobeads) over a period of 21 days of continuous operation

FIG. 14 illustrates, as shown in Example 9, the Nitrate production as a proportion of the ammonia removed in each reactor over a period of 25 days of continuous operation

FIG. 15 illustrates, as shown in Example 10, TN concentrations in the influent (NO₃ in) and effluent for reactors R37 (blank biobeads) and R38 (seeded biobeads) over a period of 21 days of continuous operation. The period of chlorine addition is shown with dotted and dashed lines.

FIG. 16: illustrates, as shown in Example 10, the specific TN removal activity for the biobeads in reactors R37 and R38 over 21 days of continuous operation. The period of chlorine addition is shown with dotted and dashed lines

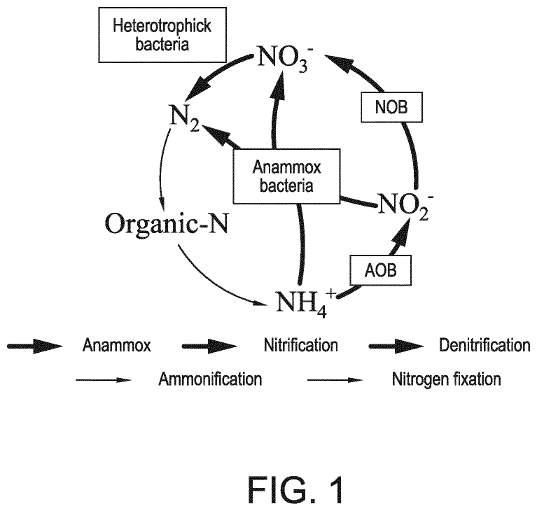

### DETAILED DESCRIPTION OF THE INVENTION

It has surprisingly been found that microorganisms survive a polymerization reaction involving crosslinking polyvinyl alcohol using the biocidal glutaraldehyde. It has furthermore surprisingly been found that the microorganisms become immobilized on the crosslinked polyvinyl alcohol and actively metabolize ammonia, nitrites and nitrates. Furthermore, it has been found that the polymerization process provides a bead-like support with a pore structure well-suited for microbial growth and for nutrient distribution.

An aspect of the invention is the replacement of chemical means to purify water by using microorganisms immobilized within an inert polymer hydrogel support, wherein the support comprises, at least in part, a biological polymer, given the support comprises the natural polysaccharide found in many forms of algae and seaweed, thus providing a biology-in-biology solution to nitrogen removal from water.

The term “community” is intended to mean a microbiological community originating from a single bacterial species or a consortium of multiple strains. Typically, a community is a microbial community, composed of a pure strain or a mixed culture, that is enhanced. The term mixed culture defines both defined (constructed by combining strains) or complex (an enriched community).

With regards to wastewater treatment, the presence of nitrogen in any form in treated effluent can be harmful to the environment the treated wastewater is being discharged to. Ammonium is directly toxic to fish and other aquatic lifeforms, while excessive levels of nitrate and nitrite can lead to eutrophication in water courses, leading to fish die offs, odours and other environmental issues.

The term microbial load is intended to mean the amount of microorganisms as determined in grams of microorganism per kg of polymer based upon the weight of the polymer support when fully hydrated.

According to the invention the term “wastewater” includes industrial wastewater, municipal wastewater, run-off from landfills, drainage of agricultural land, drainage from fish farms/aqua culture.

According to the invention the term “drinking water” includes water intended for use in municipal drinking water, including from wells, springs, lakes, rivers, ground water, surface water, lake water or any fresh water source of water.

### Preparation of Polymeric Support of Immobilized Microorganisms

The polymeric support of immobilized microorganism is typically prepared in a process comprising a first step comprising a pre-bead formation and second step comprising polyvinyl alcohol linking.

In a suitable embodiment, the polymer material comprises polyvinyl alcohol wherein the polyvinyl alcohol is a blend of polyvinyl alcohol of different molecular weights (MW), such as a blend of 2, 3, 4 or 5 PVA types, each with a MW from approximately 75.000 to approximately 225.000, such as a MW of approximately 95.000 to approximately 205.000, such as a PVA blend comprising a PVA selected from the group consisting of a PVA with a MW of approximately 125.000, PVA with a MW of approximately 145.000, and PVA with a MW of approximately 195.000.

The pre-bead formation step comprises combining sodium alginate, polyvinyl alcohol (PVA) and the microorganisms. The mixture is then added into a divalent cation-containing solution, such Ca²⁺-containing. Typically, the mixture is added in a dropwise fashion to the Ca²⁺-containing solution. Without being bound to particular theory, it is believed that the alginate and Ca²⁺ rapidly form a complex resulting in a gelate structure dispersed within the Ca²⁺-containing solution forming a heterogenous solution. Still within the theory, it is believed that the non-crosslinked PVA and microorganisms are temporarily trapped within said gelate structure but leach out of gel and into the Ca²⁺-containing solution. The PVA is thought to leach out at a higher rate than the microorganisms. The heterogenous solution comprises a gelate structure of an alginate-Ca²⁺ complex within the Ca²⁺-containing solution and with microorganisms and PVA loosely entrapped within the gelate structure. The Ca²⁺-containing solution typically further comprises PVA and microorganisms.

The alginate-Ca²⁺ complex comprises physical cross-linking, which relies on Ca²⁺ cross-linking between alginate chains.

The pre-bead formation step comprises combining sodium alginate, polyvinyl alcohol (PVA) and the microorganisms to form a mixture to be dripped into the Ca²⁺-containing solution. In the sodium alginate, polyvinyl alcohol (PVA) and microorganism-containing solution, the polyvinyl alcohol (PVA) is suitably present at a concentration 3%-15% wt/wt, such as 5-10% wt/wt, such as 5%, 6%, 7%, 8%, 9% or 10%. The alginate is typically present at a concentration of 0.25-5% wt/wt, such as 0.5-2%, such as 0.5%, 1%, 1.5% or 2%.

The pre-bead formation step comprises combining sodium alginate, polyvinyl alcohol (PVA) and the microorganisms to form mixture. A stock broth solution of microorganisms comprising from 200 g of cells/L to 1.000 g of cells/L, such as 100 to 500 g of cells/L is typically used. Typically, the stock broth solution is diluted, said diluted solution comprising to 100 to 500 g of cells/L, such as 50 to 250 g of cells/L, such as 50 g/L, 100 g/L, 125 g/L, 150 g/L, 200 g/L, and 250 g/L.

The microorganisms are preferably selected from the group consisting of ammonium oxidizing microorganisms, nitrite oxidizing microorganisms, denitrifying microorganisms, combinations thereof and anammox bacteria. The microorganisms may be selected from a mixed or pure culture of nitrite-oxidizing bacteria, a mixed or pure culture of ammonium oxidizing bacteria, a mixed or pure culture of ammonium oxidizing and nitrite-oxidizing bacteria, and a mixed or pure culture of anammox bacteria.

In suitable embodiment, the microorganisms may be selected from the group consisting of Pseudomonas lini, Pseudomonas nitroreducens, Paracoccus pantotrophus, Castellaniella defragans, Pseudomonas proteolytica, Paracoccus versutus, Paracoccus denitrificans Nitrosomonas eutropha, Nitrosomonas europaea, and Nitrobacter winogradsky,

In a suitable embodiment, the microorganisms are selected from the group consisting of a combination of Nitrosomonas eutropha and a Nitrobacter, and a combination of a Nitrosomonas europaea and a Nitrobacter.

In a further embodiment, the microorganisms are a combination of a Nitrosomonas and Nitrobacter winogradsky. In an alternative embodiment, the microorganisms are selected from the group consisting of a combination of Nitrosomonas eutropha and Nitrobacter winogradskyi, and a combination of Nitrosomonas europaea and Nitrobacter winogradskyi, preferably a combination of Nitrosomonas eutropha and Nitrobacter winogradskyi.

In an alternative embodiment wherein the immobilized microorganisms comprise ammonia oxidizing bacteria selected from Nitrosomonas spp., Nitrosococcus spp., Nitrosospira spp., Nitrosolobus spp., and Nitrosovibrio spp, and comprise nitrite oxidizing bacteria selected from Nitrobacter spp., Nitrococcus spp., Nitrospira spp., and Nitrospina spp.

This diluted stock solution is used to form a mixture comprising of sodium alginate, polyvinyl alcohol (PVA) and the microorganisms. The microorganisms are suitably present in the mixture at a concentration of 10 g/L to 500 g/L, such as 20 g/L to 80 g/L, typically from 20 g/L to 60 g/L.

The pre-bead formation step comprises combining sodium alginate, polyvinyl alcohol (PVA) and the microorganisms. The mixture is then added to a divalent or trivalent cation-containing solution, such as a divalent-containing solution, such as a Ca²⁺-containing solution. Typically, the mixture is added in a dropwise fashion to the divalent cation-containing solution, such as to the Ca²⁺-containing solution. The divalent or trivalent cation-containing solution, such as the Ca²⁺-containing solution typically comprises a dissolved salt such CaCl₂), SrCl₂, BaCl₂ or Al₂(SO₄)₃. The divalent cation containing-solution, such as the Ca²⁺-containing solution may have a cation concentration, (wt/wt) such as calcium concentration (wt/wt) ranging from 0.1% to 10%, typically from 0.5% to 5%, such as 0.5% to 2%, such as 0.5%, 1%, 1.5%, or 2%.

The second step is a cross-linking step comprises adding the gelate structure of the alginate-Ca²⁺ complex is added to a cross-linking solution comprising glutaraldehyde so as to provide a cross-linked polymer support of immobilized microorganisms. Suitably, the cross-linking solution further comprises an acid, such as sulfuric acid, H₂SO₄ or hydrochloric acid, HCl. adjusting to same pH the physical properties like elasticity, visual appearance or sphericity of the beads change along.). The cross-linking solution has an acidic pH, such as a pH of 1-5, typically 1.5 to 3.5 such as 2 or 3. The cross-linking solution when comprising sulfuric acid typically has a pH of 1.5 to 3.5 such as 2 or 3. In this step, PVA and glutaraldehyde form covalent bonds. Typically, the cross-linking solution further comprises a catalytic agent such as a sulphate, typically sodium sulphate, ammonium sulphate or potassium sulphate. Without being bound to a particular theory, the catalytic agent such as sodium sulphate acts performs at least two functions. It forms a complex with the hydroxyl groups of PVA, thereby interlinking PVA strands and/or two different positions within a PVA strand. This function supports the cross-linking with glutaraldehyde. It is furthermore thought that the catalytic agent catalyses the reaction of the aldehyde units of glutaraldehyde with the alcohol groups.

Without being bound to a particular theory, during the cross-linking step, the alginate is hydrolysed under acidic pH conditions. The hydrolysed alginate washes out, at least in part from the cross-linked polymer support of immobilized microorganisms.

Without being bound to a particular theory, it is thought that the microorganisms are protected from the harsh conditions of the cross-linking solution, namely the low pH and the presence of high amounts of the biocide glutaraldehyde by being trapped within the gelated alginate-Ca²⁺ complex.

The molar ratio of glutaraldehyde to polyvinyl alcohol plays a role in the cross-linking density of the polymer. Typically, the molar ratio of glutaraldehyde to polyvinyl alcohol is suitably from 1:10⁵ to 1:10¹⁰, such as from 1:10⁶ to 1:10⁹, such as 1:10⁷ to 1:10⁹, such as in the order of 1:10⁷, 1:10⁸ or 1:10⁹, such as in the order 1:10⁸.

In a suitable embodiment, the concentration of glutaraldehyde in the cross-linking step is less than 0.3%, such as from 0.02% to 0.25%, preferably from 0.02% to 0.2%, such as 0.05%, 0.10%, 0.15% and 0.20%.

In a suitable embodiment, the concentration of sulfuric acid (H₂SO₄) is from 0.2% to 1.0% (g/g), such as 0.3% to 0.9%, such as 0.3%, 0.4%, 0.5%, 0.6%. 0.7%, or 0.8%, typically 0.4% to 0.6%, such as 0.4%, 0.5% or 0.6%.

The reaction time of the cross-linking step is typically from 1 to 6 hours, typically 2 to 5 hours, such as 2 to 4 hours, such as 2.5 to 3.5 hours, such as 3 hours.

After the reaction time, the polymer support of immobilized microorganisms is washed in washing step in aqueous medium, such as an alkaline buffer, such as a carbonate buffer or a phosphate buffer. The buffer is typically at a pH of 7 to 10, typically pH 7.5 to 10, such as pH 8 to 10, such as 8.5, 9, 9.5 or 10, such as 8.5, 9 or 9.5, such as pH 9. The washing step may comprise the use of an alkaline buffer or the use of an amine-rich solution such as a poly-ethyleneimine solution.

The washing step removes, at least in part the unreacted glutaraldehyde. The washing step may further wash out, at least in part, the alginate. The washing step may be repeated 1 to 5 times, typically 3 times.

In some embodiments, at least some alginate may remain within the polymer support of immobilized microorganisms. The alginate entrapped within the polymer support of immobilized microorganisms does not impact performance. In an embodiment, the polymer support of immobilized microorganisms, after multiple washing, may comprise alginate. In an embodiment, the polymer support of immobilized microorganisms does not comprise alginate.

Unlike the alginate-Ca²⁺ complex which comprised physical cross-linking, relying on Ca²⁺ cross-linking between alginate chains, polymer support of immobilized microorganisms comprises polyvinyl alcohol polymeric chains covalently cross-linked via glutaraldehyde linkages.

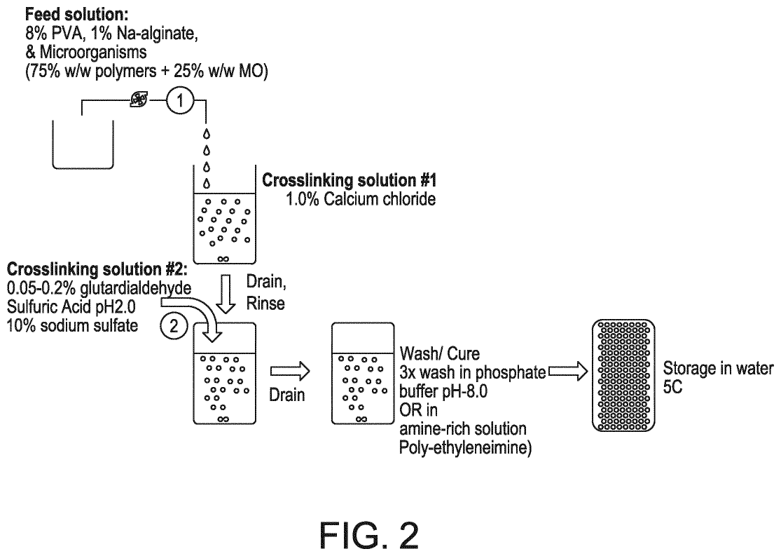

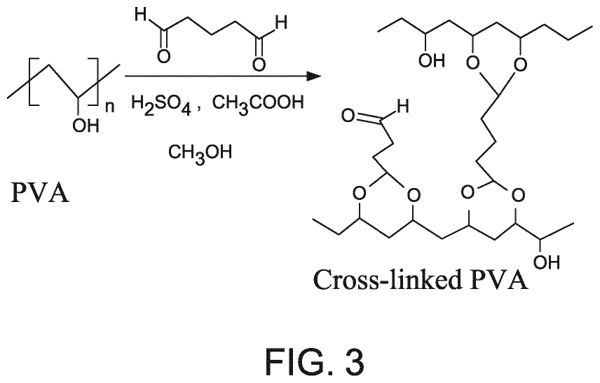

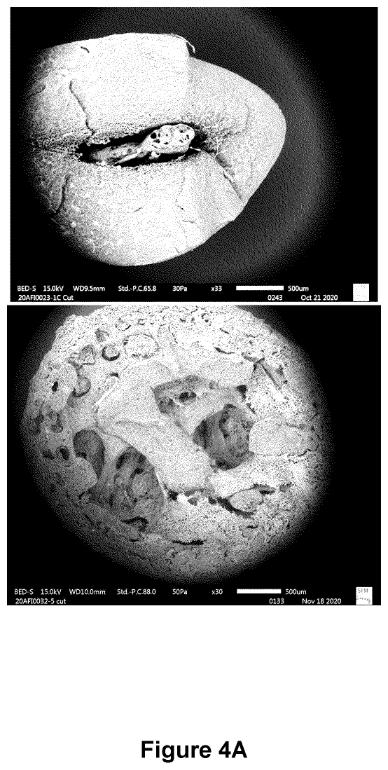

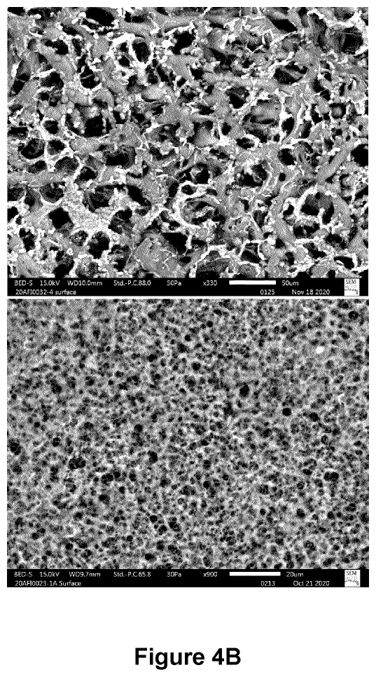

A summary of an embodiment of the method for the preparation of a polymer support of immobilized microorganisms is illustrated in FIG. 2. A solution of PVA and Na-alginate and microorganisms (75% w/w polymer+25% w/w microbes) is added to a cross-linking solution comprising calcium chloride. The gelate-containing heterogeneous solution is drained and rinsed and added to a second cross-linking solution comprising 0.05 to 2% glutaraldehyde, sulfuric acid, pH 2.0, and 10% sodium sulfate. The solution is mixed for approximately 3 hours, then drained, washed and cured in phosphate buffer pH 8.0 or in amine-rich solution such as poly-ethyleneimine. The resultant beads are stored in water at 5° C.

One aspect of the invention is directed to a method of preparing a polymer support of immobilized microorganisms, said method comprising
1a. Combining in solution a source of alginate, polyvinyl alcohol, and microorganisms into a mixture
1b. Adding said mixture to a divalent or trivalent cation-containing solution, such as a divalent cation-containing solution, such as Ca²⁺-containing solution, forming a heterogenous solution comprising a gelate structure;
2a. Adding said gelate structure to a cross-linking solution, said cross-linking solution comprising glutaraldehyde.
2b. Collecting the resultant polymer support of immobilized microorganisms

Suitably the cross-linking solution is an acidic medium. Suitably, the cross-linking solution further comprises an acid, such as sulfuric acid. Suitably the cross-linking solution further comprises a catalytic agent, such as sodium sulphate. Typically, the method further comprises
3. washing the resultant polymer support of immobilized microorganisms in aqueous medium.

### The Polymer Support

In one aspect, the invention is directed to a polymer support for immobilizing microorganisms, wherein the polymer support is a polymer hydrogel, said hydrogel comprising polyvinyl alcohol polymeric chains cross linked with glutaraldehyde; and water or an aqueous solution. It has surprisingly been found that the microorganisms become immobilized on the crosslinked polyvinyl alcohol and actively metabolize nitrogen sources including metabolize ammonia, nitrites and nitrates. The polymer support for immobilizing microorganisms, is suitable for, suited for, and/or intended for immobilizing microorganisms. Furthermore, it has been found that the polymer support comprises a pore structure well-suited for microbial growth and for nutrient distribution.

A further aspect of the invention is hence directed to a polymer support of immobilized microorganisms wherein the polymer support comprises said microorganisms immobilized within a polymer hydrogel; wherein the polymer hydrogel comprises polyvinyl alcohol polymeric chains cross linked with glutaraldehyde; and water or an aqueous solution.

It has surprisingly been found that the microorganisms become immobilized on the crosslinked polyvinyl alcohol and actively metabolize nitrogen sources including metabolize ammonia, nitrites and nitrates. Furthermore, it has been found that the polymer support comprises a pore structure well-suited for microbial growth and for nutrient distribution.

In one embodiment, the invention is directed to a polymer support of immobilized microorganisms wherein the polymer support comprises said microorganisms immobilized within a polymer hydrogel; wherein polymer hydrogel comprises cross-linked polymeric material, and water or aqueous medium; wherein the cross-linked polymeric material is a cross-linked polymer comprising polyvinyl alcohol and glutaraldehyde. The polymer support of immobilized microorganisms comprises a covalent bond between glutaraldehyde and polyvinyl alcohol polymeric chains. The covalent bond crosslinks the polyvinyl alcohol polymeric chains.

The polyvinyl alcohol polymeric chains will vary in length. Typically, the polyvinyl alcohol polymeric chains consist of 500-3.000 monomer units and a molecular weight of 22.000 g/mol to 130.000 g/mol. The polyvinyl alcohol polymeric chains are cross-linked with glutaraldehyde. The crosslink may comprise monomeric, dimeric, oligomeric, or polymeric forms of glutaraldehyde.

The crosslinking density, determined as the number of glutaraldehyde crosslinking units, may affect morphological properties of the polymer support. In one embodiment, the crosslinking density, determined as the number of glutaraldehyde crosslinking units, is 8 to 25%, such as 10 to 20%, such as 12-20%, such as about 12%, 13%, 15%, 16%, 17%, 18%, 19% or 20%.

The polymer support of immobilized microorganisms is, as stated, a polymer hydrogel. Typically, the hydrogel comprises from 60 to 99 wt % cross-linked polymeric material and 1 to 40 wt % water or aqueous medium, such as from 60 to 98 wt % cross-linked polymeric material and 2 to 40 wt % water or aqueous medium, such as from 65 to 95 wt % cross-linked polymeric material and 5 to 35 wt % water or aqueous medium, such as from 65 to 90 wt % cross-linked polymeric material and 10 to 35 wt % water or aqueous medium, such as from 65 to 85 wt % cross-linked polymeric material and 15 to 35 wt % water or aqueous medium, such as from 65 to 80 wt % cross-linked polymeric material and 20 to 35 wt % water or aqueous medium, or such as from 65 to 75 wt % cross-linked polymeric material and 25 to 35 wt % water or aqueous medium.

In a further embodiment, the polymer support of immobilized microorganisms further comprises alginate in monomer or polymeric form, entangled with cross-linked polyvinyl alcohol polymeric chains, such as so as to form an interpenetrating polymer network or semi-interpenetrating polymer network.

The polymer support comprises pores. The polymer support is a porous structure. The immobilized microorganisms are immobilized within the polymer support on the surface of the pores. Entrapment of the microorganisms in the inner matrix provides an advantage of the method in that it serves to physically protect immobilized cells. Cell attachment or adsorption of microorganisms to the inner matrix of polymer support may be by weak (non-covalent), generally non-specific interactions such as electrostatic interactions.

In a typical embodiment, the polymer support is adequately porous to allow the substrate to diffuse into the polymer hydrogel support and the products or metabolites to diffuse out. In a typical embodiment, a central volume of the polymer support comprises one or more macropores, and major volume of the polymer support comprises micropores. Without being bound to a particular theory, the central macropores allows for convection, which is an efficient method of mass transfer without applying pressure.

In a typical embodiment, the pores of the polymer support are non-uniform in size. In one embodiment, an inner central fraction of the bead volume comprises macropores, whereas an outer fraction of the bead volume comprises micropores. The term macropores is intended to mean pores with an average size of at least 100 microns. The term micropore is intended to mean pores with an average pore size of less than 100 microns.

Defined alternatively, in an embodiment, the core of polymer support is devoid of polymer, such as a core of at least 100 microns in longest diameter, such as at least 200 microns, typically from 100-2.000 microns, such as 200-2.000 microns, such as 200-1.500 microns, such as 100-1.000 microns, such as 200-1.000 microns

In one embodiment, the polymer support comprises pores having a gradient pore size in that inner portion of the volume of the polymer support has a larger pore size than average pore size if the remaining volume of the polymer support.

In one embodiment of the invention, the polymer support comprises pores having a gradient pore size in that the outer one-third of the polymer support has pores of a smaller average diameter than the pore size of the middle third of the polymer support, which in turn has a smaller average diameter than the pore size of the inner one-third of the polymer support. Within this embodiment, the outer one-third of the polymer support has an average pore diameter from 5 to 20 microns. The middle third of the polymer support has a larger average pore diameter than the outer one-third. It a suitable embodiment, it has an average pore diameter from 10 to 100 microns. The inner one-third of the polymer support has, in an embodiment, a larger average pore diameter than the middle third of the polymer support. The inner third of the polymer support has, in one embodiment, an average pore diameter from 100-2.000 microns. In one embodiment, at least 50% of the volume of the inner third is a cavity. The cavity is a volume within the polymer support that is substantially free from cross-linked polyvinyl alcohol. The center of the polymer support may comprise, in its center, a cavity having volume comprising 50-100% of the volume of the inner one third of the polymer support. A cavity is a volume free from cross-linked polyvinyl alcohol. Without being bound to a particular theory, the cavity serves as a central distribution center for nutrients, metabolites and substrate for the microorganisms, allowing for flow and distribution within the polymer support.

The immobilized microorganisms grow within the polymer hydrogel. That is to say that the microorganisms grow on the surface of the pores. The inert polymer hydrogel retains the microorganism, albeit not irreversibly in that a fraction of the population of the microorganisms may leak out of the polymer support by detaching from the inner matrix of the polymer support and leaking through the pores to exit the polymer support. In an embodiment of the invention, the outer surface of the polymer support does not comprise a skin or shell, which may serve to retain the cells or metabolites by means of having a smaller pore diameter than the outer third of the polymer support.

The polymer support of the invention is resistant to dissolution in water. It is suitable to be re-used or reusable.

In one embodiment, the polymeric support is chemically substantially uniform in that the surface, body and core of the carrier is made of the same chemical components. However, as known to the person skilled in the art, due to different rates and extent of curing at the surface compared to within the body of the carrier, during the preparation of the polymer, some physio-chemical properties on the surface may differ with the physio-chemical properties within the core and throughout the hydrogel. Accordingly, there may be different degrees of crosslinking at the surface. However, according to the invention, these differences do not constitute a shell or coating or fibrous network on the surface.

In one embodiment, the invention is directed to a polymer support of immobilized microorganisms wherein the polymer support comprises said microorganisms immobilized within a polymer hydrogel; wherein polymer hydrogel comprises cross-linked polymeric material, alginate and water or aqueous medium; wherein the cross-linked polymeric material is a cross-linked polymer comprising polyvinyl alcohol and glutaraldehyde. In some embodiments, at least some alginate may remain within the polymer support of immobilized microorganisms. The alginate entrapped within the polymer support of immobilized microorganisms does not negatively impact performance of the polymer support. In an embodiment, the polymer support of immobilized microorganisms, after multiple washing, may comprise alginate. In an embodiment, the polymer support of immobilized microorganisms does not comprise alginate.

The polymer support comprises glutaraldehyde cross-linking PVA chains. An increased content of glutaraldehyde results in more PVA hydroxyl groups consumed and more acetal rings and ether linkages formed as a result of the crosslink formation. Furthermore, the presence of an acid catalyst in the preparation of the polymer support, such as sulfuric acid, also increases crosslink formation. Cross-link formation increases mechanical strength of the polymer support. Increased mechanical strength is observed with increased degree of crosslinking. With the increasing content of GA, the polymer support rigidity increases. The polymer support is elastic and malleable with excellent mechanical properties. The elasticity modulus is typically between 1.4 and 2.2 GPa, such as between 1.5 and 2 GPa. The tensile strength is typically between 3 and 6 MPa.

In one aspect, the invention is directed to a polymer support of immobilized microorganisms wherein the polymer support comprises said microorganisms immobilized within a polymer hydrogel. The concentration of microorganisms within the polymer hydrogel, known as the microbial load, is typically in the range of 5 g/kg bead to 250 g/kg, typically 10 g/kg to 150 g/kg.

The cell density of the microorganisms can be tailored to the type of microorganisms, intended metabolic activity and whether the polymer support is intended for use for cleaning ground water, spring water, drinking water or wastewater. In one embodiment of the invention, a high microbial load of immobilized microorganisms within the polymer support may be used, in some embodiments, to improve the product yield and the volumetric productivity of the bioreactors. The microbial load in the support is suitably at a concentration of at least about 5 grams, such as at least 10 grams/kg, such as at least 20 grams/kg, such as at least 50 grams/kg

In a suitable embodiment of the polymer support of immobilized microorganisms, the microorganisms are selected from a mixed or pure culture of nitrite-oxidizing bacteria, a mixed or pure culture of ammonium oxidizing bacteria, a mixed or pure culture of ammonium oxidizing and nitrite-oxidizing bacteria, a mixed or pure culture of denitrifying bacteria and a mixed or pure culture of anammox bacteria. In a typical embodiment, the microorganisms are a combination of ammonium oxidizing microorganisms and nitrite oxidizing microorganisms. In a preferred embodiment, the microorganisms are a combination of the ammonia oxidizing bacteria Nitrosomas spp. and the nitrite oxidizing microorganisms Nitrobacter spp.

The polymer support of immobilized microorganisms may be of any shape but is typically according to spherical, oval, elliptical, bead-shaped, oblong, cylindrical, or capsule-like in shape. The polymer support is typically 1 to 10 mm long at its longest axis, typically 2 to 8 mm, such as 3 to 7 mm or 3 to 6 mm. In the axis perpendicular to the longest axis, the polymer support may be 1 to 10 mm, typically 2 to 8 mm, such as 3 to 7 mm or 3 to 6 mm. Typically, the aspect ratio is from 0.5 to 1, such as 0.5, 0.6, 0.7, 0.8, 0.9 or 1.

The polymer support of immobilized microorganisms is typically spherical or bead-shaped having a diameter of 1 to 10 mm, typically 2 to 8 mm, such as 3 to 7 mm or 3 to 6 mm with an aspect ratio from 0.6 to 1, typically from 0.8 to 1.

### The Microorganisms

In one aspect, the invention is directed to a polymer support of immobilized microorganisms wherein the polymer support comprises said microorganisms immobilized within a polymer hydrogel; wherein the polymer hydrogel comprises polyvinyl alcohol polymeric chains cross linked with glutaraldehyde. The microorganisms immobilized within the polymer hydrogel may be selected from the group consisting of ammonia oxidizing bacteria, nitrite oxidising bacteria, heterotrophic bacteria and anaerobic ammonium-oxidizing bacteria.

In typical embodiment, the microorganisms immobilized within the polymer hydrogel may be selected from the group consisting of Nitrosomonas spp., Nitrobacter spp., Nitrosococcus spp., Nitrosospira spp., Nitrosolobus spp., and Nitrosovibrio spp., Nitrotoga spp., Nitrospira spp. Pseudomonas spp., Paracoccus spp., Hyphomicrobium spp., Castellaniella spp., Janthinobacterium spp., Acidovorax spp., Aeromonas spp., Cellulomonas spp., Buttiauxella spp., Microvirgula spp., Klebsiella spp., Shewanella spp., Pelosinus spp., Variovorax spp., Hydrogenophaga spp., Raoultella spp., Bacillus spp., Achromobacter spp., Ochrobactrum spp., Flavobacterium spp., and Delftia. In a more typical embodiment, the microorganisms immobilized within the polymer hydrogel may be selected from the group consisting of Nitrosomonas spp., Nitrobacter spp, Nitrospira spp. Nitrosococcus spp., Nitrosospir a spp., Nitrosolobus spp., and Nitrosovibrio spp., Pseudomonas spp., Paracoccus spp., Castella niella spp., Hyphomicrobium spp., Ochrobactrum spp., and Janthinobacterium spp. In a preferred embodiment, the microorganisms immobilized within the polymer hydrogel may be selected from the group consisting of Nitrosomonas spp., Nitrobacter spp., Nitrospira spp., Nitrosococcus spp., Paracoccus spp., and Pseudomonas spp.

In suitable embodiment, the microorganisms immobilized within the polymer hydrogel may be selected from the group consisting of Paracoccus pantotrophus, Paracoccus versutus, Paracoccus denitrificans, Castellaniella defragrans, Pseudomonas proteolytica, Pseudomonas alcaliphila, Pseudomonas chlororaphis, Pseudomonas monteilii, Pseudomonas lini, Pseudomonas alkylphenolica, Pseudomonas nitroreducens, Pseudomonas stutzeri, Ochrobactrum anthropic, Ochrobactrum intermedium, Aeromonas media, Aeromonas veronii, Aeromonas jandaei, Aeromonas hydrophila, Cellulomonas chitinilytica, Cellulomonas cellasea, Cellulomonas hominis, Flavimobis soli, Flavobacterium banpakuense, Buttiauxella agrestis, Buttiauxella noackiae, Achromobacter denitrificans, Pelosinus fermentans, Variovorax dokdonensis, Hydrogenophaga bisanensis, Raoultella terrigena, Delftia lacustris, Shewanella putrefaciens, Acidovorax soli, Hyphomicrobium denitrificans. Nitrosomonas eutropha, Nitrosomonas europaea, Nitrobacter winogradsky, Microvirgula aerodenitrificans, Candidatus kuenenia, Candidatus brocadia, Candidatus anammoxoglobus, Candidatus jettenia, and Candidatus scalindua

In a typical embodiment, the microorganisms immobilized within the polymer hydrogel may be selected from the group consisting of Paracoccus pantotrophus, Paracoccus versutus, Paracoccus denitrificans, Castellaniella defragrans, Pseudomonas proteolytica, Pseudomonas alcaliphila, Pseudomonas chlororaphis, Pseudomonas monteilii, Pseudomonas lini, Pseudomonas alkylphenolica, Pseudomonas nitroreducens, Ochrobactrum anthropic, Ochrobactrum intermedium, Aeromonas veronii, Cellulomonas chitinilytica, Cellulomonas cellasea, Cellulomonas hominis, Flavimobis soli, Achromobacter denitrificans, Pelosinus fermentans, Acidovorax soli, Hyphomicrobium denitrificans, Microvirgula aerodenitrificans, Nitrosomonas eutropha, Nitrosomonas europaea, and Nitrobacter winogradsky.

In preferred embodiment, the microorganisms immobilized within the polymer hydrogel may be selected from the group consisting of Paracoccus pantotrophus, Paracoccus versutus, Paracoccus denitrificans, Castellaniella defragrans, Pseudomonas lini, Pseudomonas proteolytic, Pseudomonas alkylphenolica, Nitrosomonas eutropha, Nitrosomonas europaea, and Nitrobacter winogradsky.

In an embodiment, the microorganisms immobilized within the polymer hydrogel are selected from the group consisting of Pseudomonas lini, Paracoccus pantotrophus, Paracoccus pandtodrophus, Castellaniella defragans, Pseudomonas proteolytica, Paracoccus versutus, Paracoccus denitrificans, Nitrosomonas eutropha, Nitrosomonas europaea, and Nitrobacter winogradsky,

In an embodiment, the microorganisms are selected from the group consisting of a combination of Nitrosomonas eutropha and Nitrobacter winogradskyi, and a combination of Nitrosomonas europaea and Nitrobacter winogradskyi, preferably a combination of Nitrosomonas eutropha and Nitrobacter winogradskyi.

In an embodiment, the microorganisms are selected from the group consisting of Pseudomonas lini, Paracoccus pantotrophus, Paracoccus versutus and combinations thereof.

In one aspect, the invention is directed to a polymer support of immobilized microorganisms wherein the polymer support comprises said microorganisms immobilized within a polymer hydrogel; wherein the polymer hydrogel comprises polyvinyl alcohol polymeric chains cross linked with glutaraldehyde; and water or an aqueous solution; wherein the immobilized microorgansims comprise Pseudomonas linil.

In one aspect, the invention is directed to a polymer support of immobilized microorganisms wherein the polymer support comprises said microorganisms immobilized within a polymer hydrogel; wherein the polymer hydrogel comprises polyvinyl alcohol polymeric chains cross linked with glutaraldehyde; and water or an aqueous solution; wherein the immobilized microorgansims comprise Paracoccus pantotrophus.

In one aspect, the invention is directed to a polymer support of immobilized microorganisms wherein the polymer support comprises said microorganisms immobilized within a polymer hydrogel; wherein the polymer hydrogel comprises polyvinyl alcohol polymeric chains cross linked with glutaraldehyde; and water or an aqueous solution; wherein the immobilized microorgansims comprise Castellaniella defragans.

In one aspect, the invention is directed to a polymer support of immobilized microorganisms wherein the polymer support comprises said microorganisms immobilized within a polymer hydrogel; wherein the polymer hydrogel comprises polyvinyl alcohol polymeric chains cross linked with glutaraldehyde; and water or an aqueous solution; wherein the immobilized microorgansims comprise Pseudomonas proteolytica.

In one aspect, the invention is directed to a polymer support of immobilized microorganisms wherein the polymer support comprises said microorganisms immobilized within a polymer hydrogel; wherein the polymer hydrogel comprises polyvinyl alcohol polymeric chains cross linked with glutaraldehyde; and water or an aqueous solution, wherein the immobilized microorgansims comprise Paracoccus versutus.

In one aspect, the invention is directed to a polymer support of immobilized microorganisms wherein the polymer support comprises said microorganisms immobilized within a polymer hydrogel; wherein the polymer hydrogel comprises polyvinyl alcohol polymeric chains cross linked with glutaraldehyde; and water or an aqueous solution; wherein the immobilized microorgansims comprise Paracoccus denitrificans.

In one aspect, the invention is directed to a polymer support of immobilized microorganisms wherein the polymer support comprises said microorganisms immobilized within a polymer hydrogel; wherein the polymer hydrogel comprises polyvinyl alcohol polymeric chains cross linked with glutaraldehyde; and water or an aqueous solution, wherein the immobilized microorgansims comprise Nitrosomonas eutropha.

In one aspect, the invention is directed to a polymer support of immobilized microorganisms wherein the polymer support comprises said microorganisms immobilized within a polymer hydrogel; wherein the polymer hydrogel comprises polyvinyl alcohol polymeric chains cross linked with glutaraldehyde; and water or an aqueous solution wherein the immobilized microorgansims comprise Nitrosomonas europaea.

In one aspect, the invention is directed to a polymer support of immobilized microorganisms wherein the polymer support comprises said microorganisms immobilized within a polymer hydrogel; wherein the polymer hydrogel comprises polyvinyl alcohol polymeric chains cross linked with glutaraldehyde; and water or an aqueous solution wherein the immobilized microorgansims comprise Nitrobacter winogradsky.

In a suitable embodiment, the microorganisms are selected from the group consisting of a combination of Nitrosomonas eutropha and a Nitrobacter, and a combination of a Nitrosomonas europaea and a Nitrobacter. In a further embodiment, the microorganisms are a combination of a Nitrosomonas and Nitrobacter winogradsky. In an alternative embodiment, the microorganisms are selected from the group consisting of a combination of Nitrosomonas eutropha and Nitrobacter winogradskyi, and a combination of Nitrosomonas europaea and Nitrobacter winogradskyi, preferably a combination of Nitrosomonas eutropha and Nitrobacter winogradskyi.

In an alternative embodiment, wherein the immobilized microorganisms comprise ammonia oxidizing bacteria selected from Nitrosomonas spp., Nitrosococcus spp., Nitrosospira spp., Nitrosolobus spp., and Nitrosovibrio spp, and comprise nitrite oxidizing bacteria selected from Nitrobacter spp., Nitrococcus spp., Nitrospira spp., and Nitrospina spp.

The microorganisms may be selected from the group consisting of ammonia oxidizing bacteria. Ammonia oxidizing bacteria (AOB) play a critical role in the global nitrogen cycle and the removal of nitrogen from wastewater treatment plants (WWTPs) through their oxidization of ammonia (NH₃) to nitrite (NO₂⁻). The oxidation of NH3 is a two-step process in which NH₃ is oxidized, via the ammonia monooxygenase (AMO) enzyme, to hydroxylamine (NH₂OH), which is further oxidized to NO₂⁻, via the hydroxylamine oxidoreductase (HAO) enzyme.

Typically, ammonia-oxidizing bacteria (AOB) may be selected from Nitrosomonas and Nitrosococcus.

The microorganisms may be selected from the group consisting of heterotrophic bacteria. Heterotrophic bacteria may be selected from the group consisting of Paracoccus pantotrophus, Paracoccus versutus, Paracoccus denitrificans, Castellaniella defragrans, Pseudomonas proteolytica, Pseudomonas alcaliphila, Pseudomonas chlororaphis, Pseudomonas monteilii, Pseudomonas lini, Pseudomonas alkylphenolica, Pseudomonas nitroreducens, Pseudomonas stutzeri, Ochrobactrum anthropic, Ochrobactrum intermedium, Aeromonas media, Aeromonas veronii, Aeromonas jandaei, Aeromonas hydrophila, Cellulomonas chitinilytica, Cellulomonas cellasea, Cellulomonas hominis, Flavimobis soli, Flavobacterium banpakuense, Buttiauxella agrestis, Buttiauxella noackiae, Achromobacter denitrificans, Pelosinus fermentans, Variovorax dokdonensis, Hydrogenophaga bisanensis, Raoultella terrigena, Delftia lacustris, Shewanella putrefaciens, Acidovorax soli, Hyphomicrobium denitrificans, Microvirgula aerodenitrificans, Candidatus kuenenia, Candidatus brocadia, Candidatus anammoxoglobus, candidatus jettenia, and Candidatus scalindua

Heterotrophic bacteria may be typically selected from the group consisting of Paracoccus pantotrophus, Paracoccus versutus, Paracoccus denitrificans, Castellaniella defragrans, Pseudomonas proteolytica, Pseudomonas alcaliphila, Pseudomonas chlororaphis, Pseudomonas monteilii, Pseudomonas lini, Pseudomonas alkylphenolica, Pseudomonas nitroreducens, Ochrobactrum anthropic, Ochrobactrum intermedium, Aeromonas veronii, Cellulomonas chitinilytica, Cellulomonas cellasea, Cellulomonas hominis, Flavimobis soli, Achromobacter denitrificans, Pelosinus fermentans, Acidovorax soli, Hyphomicrobium denitrificans, and Microvirgula aerodenitrificans.

Heterotrophic bacteria may be preferably selected from the group consisting of Paracoccus pantotrophus, Paracoccus versutus, Paracoccus denitrificans, Castellaniella defragrans, Pseudomonas lini, Pseudomonas proteolytic, and Pseudomonas alkylphenolica

The denitrification process typically requires a carbon source. In one aspect of the invention, the microorganism is a denitrifier and denitrification is accompanied by the addition of a carbon source. Suitable embodiments of this aspect of the invention comprise a carbon source selected from the group consisting of methanol, ethanol, acetate, acetic acid, glycerol, glycol, molasses, corn syrup, sucrose solutions, commercially available carbon sources, fermented organic wastes, industrial wastewaters. In a more preferable embodiment consisting of methanol, ethanol, acetate, acetic acid, glycerol, commercially available carbon sources. In a most preferred embodiment, the carbon source is selected from the group consisting of methanol, glycerol or commercially available carbon sources.

In a preferable embodiment, wherein the carbon source is methanol, the microorganism may be selected from a methylotrophic bacteria, which use methanol as a carbon source, selected from the group consisting of Paracoccus pantotrophus, Paracoccus versutus, Paracoccus denitrificans, Castellaniella defragrans, Pseudomonas proteolytica, Pseudomonas alcaliphila, Pseudomonas chlororaphis, Pseudomonas monteilii, Pseudomonas lini, Pseudomonas alkylphenolica, Pseudomonas nitroreducens, Pseudomonas stutzeri, Ochrobactrum anthropic, Ochrobactrum intermedium, Aeromonas media, Aeromonas veronii, Aeromonas jandaei, Aeromonas hydrophila, Cellulomonas chitinilytica, Cellulomonas cellasea, Cellulomonas hominis, Flavimobis soli, Flavobacterium banpakuense, Buttiauxella agrestis, Buttiauxella noackiae, Achromobacter denitrificans, Pelosinus fermentans, Variovorax dokdonensis, Hydrogenophaga bisanensis, Raoultella terrigena, Delftia lacustris, Shewanella putrefaciens, Acidovorax soli, Hyphomicrobium denitrificans. Microvirgula aerodenitrificans, Candidatus kuenenia, Candidatus brocadia, Candidatus anammoxoglobus, Candidatus jettenia, and Candidatus scalindua.

In a high typical embodiment, in embodiments wherein a carbon source is to be added, the microorganism is a methylotrophic bacteria, methanol is used as a carbon source, and is selected from the group consisting of Paracoccus pantotrophus, Paracoccus versutus, Paracoccus denitrificans, Hyphomicrobium denitrificans, Pseudomonas lini, Pseudomonas chlororaphis, Pseudomonas alcaliphila and Pseudomonas alkylphenolica such as Pseudomonas lini, Paracoccus pantotrophus and Paracoccus versutus.

An advantageous feature of the microorganisms of the invention are microorganisms having a feature selected from the group consisting of a robust performance, activity at low temperatures, activity with low levels of additional carbon, selectivity for specific carbon sources. In one aspect, the invention is directed to a polymer support of immobilized microorganisms wherein the polymer support comprises said microorganisms immobilized within a polymer hydrogel; wherein the polymer hydrogel comprises polyvinyl alcohol polymeric chains cross linked with glutaraldehyde; and water or an aqueous solution, wherein has at least 80% its maximum activity at below 20° C., such as below 15° C., such as below 10° C.

### Water Treatment

An aspect of the invention is directed to improving the efficacy of biological nitrogen removal processes comprises preparing the polymer support of the invention. The polymer support of the invention enables the integration of selected and concentrated microorganisms.

The benefits of the application of immobilization of microorganisms include, but are not limited to: an ability to maintain a microbiological community that is enhanced, and stable enough to achieve the treatment goals, an ability to limit or eliminate the production of excess biomass, protection of the microorganisms from extreme operational conditions including for example pH and temperature.

An aspect of the invention is directed to a method of treating water comprising adding the polymer support of microorganisms, as defined herein. The water may be water intended for drinking, such as from a natural source of water, including from a well, a spring, lake, river, ground water, surface water, or any fresh water source of water. The water may alternatively be wastewater, such as industrial wastewater or municipal wastewater. An aspect of the invention is directed to a method of treating water, such as any of drinking water, municipal wastewater or industrial wastewater, such as any of drinking water or municipal wastewater comprising mixing said water a polymer support of microorganisms, as defined herein.

An aspect of the invention is directed to a method of reducing the amount of nitrogen-containing compounds in water comprising mixing said water with a polymer support of microorganisms, as defined herein. In one embodiment, the invention is directed to a method of reducing the levels of contaminants in water, said contaminants selected from the group consisting of ammonia, nitrate and nitrite in water comprising adding to said water a polymer support of microorganisms, as defined herein. Alternatively defined, the invention is directed, in one aspect, to a method of reducing the total nitrogen (TN) content in wastewater or in drinking water comprising adding to said water a polymer support of microorganisms, as defined herein. Total nitrogen is the sum of total nitrogen Kjeldahl nitrogen (organic N+NH3), nitrate (NO3)-nitrogen and nitrite (NO₂)-nitrogen.

The natural level of ammonia or nitrate in surface water is typically less than 5 mg/L, more typically less than 2 mg/ml or 1 mg/mL. However, the effluent of municipal wastewater treatment plants, typically have a level of ammonia or nitrate of 20 mg/mL to 50 mg/mL, such as 30 mg/L. In industrial wastewater treatment plants, the wastewater comprises from 5 to 400 mg/L of ammonia or nitrate, such as 5 to 200 mg/L, such as 5 to 100 mg/L of, such as 10 to 80 mg/l, such as 15 to 50, 20 to 40 mg/L of ammonia. A plant that discharges to rapid infiltration basins (percolation ponds) may have an effluent nitrate limit of 12 mg/L. A treatment plant discharging to a nearby stream, river or wetland may have a total nitrogen limit of 3 mg/L, or an unionized ammonia (NH₃) limit of 0.2 mg/L. Accordingly, one aspect of the invention is directed to a method of reducing the nitrate level in wastewater to less than 12 mg/L, such as less than 10 mg/mL, less than 8 mg/L, less than 6 mg/L, less than 5 mg/L, less than 4 mg/L, less than 2 mg/L, less than 1 mg/L, comprising adding to said water a polymer support of microorganisms, as defined herein.

An aspect of the invention is directed to a method of reducing the total nitrogen (TN) content in wastewater or in drinking water comprising adding to said water a polymer support of microorganisms. Suitably, the method is directed to reducing the total nitrogen level in wastewater to less than 3 mg/L, such as less than 2 mg/L or 1 mg/L.

A further aspect of the invention is directed to a method of reducing the unionized ammonia (NH₃) content in wastewater or in drinking water comprising adding to said water a polymer support of microorganisms. Suitably, the method is directed to reducing the unionized ammonia (NH₃) content to less than 0.5 mg/L, such as less than 0.4 mg/L, such as less than 0.3 mg/L, or less than 0.2 mg/L

In one embodiment, the method comprises use of the polymer support of immobilized microorganisms of the invention for reducing the levels of ammonium in wastewater by at least 80%, such as by at least 90%, preferably by at least 95%, such as by at least 98%. The wastewater, depending on its source, may comprise from 5 to 200 mg/L of ammonia, such as 5 to 200 mg/L ammonia, such as 5 to 100 mg/L of ammonia, such as 10 to 80 mg/l, such as 15 to 50, 20 to 40 mg/L of ammonia, and wherein the levels of ammonia are reduced to below 30 mg/L, such as below 10 mg/L, 5 mg/L, such as below 2, below 1 mg/L.

As can be seen from the Examples, the polymer support of immobilized microorganisms of the invention were able to demonstrate an ability to achieve 60% removal of ammonia within 5 days of operation, while at the same time achieving over 85% conversion of ammonia to nitrate. This indicates that polymer support of with nitrifiers has the potential to provide significant advantages over current technologies applied in biological nitrification processes due to the ability of the biobeads to achieve high levels of ammonia removal and conversion to nitrate within a very short period and under challenging operational conditions (high ammonia loading on the reactor). At no time during the trials, were the blank beads able to achieve the same results or performance as the seeded biobeads, further demonstrating the advantage the seeding of biobeads provides compared to traditional biofilm carrier based technologies that rely on colonization of the surface of carriers (in this case the blank biobeads) by indigenous nitrifying communities. The results indicate that there is a clear advantage to integrating a strong nitrification microbial community into a polymer support.

As can be seen from the Examples, an embodiment of the invention is directed to a method of reducing the total ammonia nitrogen (TAN) treatment from wastewater comprising adding to the wastewater a polymer support of microorganisms wherein the microorganisms are selected from Nitrosomonas eutropha and Nitrobacter winogradskyi or a combination thereof, wherein the concentration of ammonia is from 10-80 mg-N/L, such as 60 mg-N/L. The Examples demonstrate very high levels of ammonia for municipal wastewater, demonstrating the robustness of the method of the invention, wherein the microorganisms survive and are active at ammonia levels such as 60 mg-N/L.

The level of activity, the level to which the levels of ammonia are to be lowered can, at least in part, be regulated by control of microbial load and/or the bead load. The microbial load is the concentration of microorganisms immobilized within the polymer support. The bead load is the concentration of polymer supports (beads) per unit volume of the water tank, bed, pond or treatment system. In a suitable embodiment, the polymer support is combined with the water at a bead load of 5% to 30 w/v, such as 10% to 30% w/v, as 15% to 30% w/v, such as 15% w/v, 20% w/v, 25% w/v or 30% w/v. For the reducing the levels of ammonia in water, wherein the microorganisms immobilized within the polymer support are Nitrosomonas eutropha, Nitrobacter winogradskyi and combinations thereof, the microbial load may be selected from 5 g/kg bead to 250 g/kg, typically 10 g/kg to 150 g/kg, such as 10 g/kg, 20 g/kg, 30 g/kg, 40 g/kg, 50 g/kg, 60 g/kg, 70 g/kg, 90 g/kg, 100 g/kg, 110 g/kg, 120 g/kg, 130 g/kg, 140 g/kg and 150 g/kg.

In an alternative embodiment, the method of denitrifying water comprises adding to the wastewater a polymer support, wherein the microorganism is selected from the group consisting of Pseudomonas lini, Paracoccus pantotrophus and combinations thereof.

The immobilized microorganisms are typically retained in the biological treatment process through the application of settling zones, screens, filters, or with hydrocyclones. This will ensure that the immobilized microorganisms remain in the process, thereby maintaining a high rate of activity and lowering the risk of the immobilized microorganisms leaving the biological treatment process and causing a pollution risk themselves.

The two-step process of nitrogen bio-elimination from wastewater generally consists of nitrification under strict aerobic conditions followed by denitrification under anoxic conditions. Ammonia primarily present in wastewaters are being oxidized to nitrite and eventually nitrate with the help of obligate aerobic autotrophs known as ammonia-oxidizing bacteria (AOB) such as Nitrosomonas, Nitrosococcus. The NO₂⁻ to NO₃⁻ conversion is fulfilled by nitrite-oxidizing bacteria (NOB) Nitrobacter. Accordingly, the polymer support of immobilized microorganisms may be such that the ammonia oxidizing bacteria are selected from Nitrosomonas spp., Nitrosococcus spp., Nitrosospira spp., Nitrosolobus spp., and Nitrosovibrio spp, and wherein the nitrite oxidizing bacteria are selected from Nitrobacter spp., Nitrococcus spp., Nitrospira spp., and Nitrospina spp. In a preferred embodiment, the microorganisms are a combination of the ammonia oxidizing bacteria Nitrosomas spp. and the nitrite oxidizing microorganisms are Nitrobacter spp. In an embodiment of the invention, the microorganisms are selected from the group consisting of a combination of Nitrosomonas eutropha and a Nitrobacter, and a combination of a Nitrosomonas europaea and a Nitrobacter. In a related embodiment, the microorganisms are a combination of a Nitrosomonas and Nitrobacter winogradsky, such as wherein the microorganisms are selected from the group consisting of a combination of Nitrosomonas eutropha and Nitrobacter winogradskyi, and a combination of Nitrosomonas europaea and Nitrobacter winogradskyi, preferably a combination of Nitrosomonas eutropha and Nitrobacter winogradskyi.

Denitrification occurs under anoxic/anaerobic conditions. Denitrification is the sequential process involving the dissimilatory reduction of one or both the ionic nitrogen oxides, nitrate (NO₃⁻) and nitrite (NO₂) to gaseous nitrogen oxides, nitric oxide (NO), nitrous oxide (N₂O) and finally reduce to the ultimate product, dinitrogen (N₂) thus removing biologically available nitrogen and returning it to the atmosphere.

Both nitrate and nitrite are fully converted to atmospheric nitrogen. However, insufficient carbon sources, low dissolved oxygen (DO) concentrations and operational fluctuations or environmental conditions lead to improper denitrification and N₂O accumulation and emissions. One pathway to reduce N₂O production is to select the right organisms for the denitrification process.

Denitrifiers with their facultative anaerobic traits perform denitrifying activities under the presence of oxygen driving an increase in N₂O as a denitrification intermediate. Many heterotrophic nitrifiers along with the oxidation of NH₃ can simultaneously perform aerobic denitrification. N₂O is then generated. Accordingly, the judicious selection of denitrifiers is an important aspect of the present invention.

As shown by the Examples, polymer hydrogels comprising Paracoccus are a preferred embodiment in the denitrification of wastewater. Paracoccus pantotrophus grows aerobically with a large variety of carbon sources and with molecular hydrogen or thiosulfate as an energy source, and nitrate serves as electron acceptor under anaerobic conditions. The denitrification properties of Paracoccus denitrificans render it a preferred microorganism. Paracoccus denitrificans reduces nitrite to nitrogen gas while either Nitrosomonas eutropha or Nitrosomonas europaea oxidizes ammonia to nitrite, thus fueling the former metabolism. Accordingly, one embodiment comprises the combined use of Paracoccus denitrificans and either Nitrosomonas eutropha or Nitrosomonas europaea.

In one aspect of the invention, denitrification is accompanied by the addition of a carbon source. Suitable embodiments of this aspect of the invention comprise a carbon source selected from the group consisting of methanol, ethanol, acetate, acetic acid, glycerol, glycol, molasses, corn syrup, sucrose solutions, commercially available carbon sources, fermented organic wastes, industrial wastewaters. In a more preferable embodiment consisting of methanol, ethanol, acetate, acetic acid, glycerol, commercially available carbon sources. In a most preferable embodiment consisting of methanol, glycerol or commercially available carbon sources. In a preferred embodiment, the carbon source is methanol, the microorganism is selected from a methylotrophic bacteria, which use methanol as a carbon source, selected from the group consisting of Paracoccus pantotrophus, Paracoccus versutus, Paracoccus denitrificans, Castellaniella defragrans, Pseudomonas proteolytica, Pseudomonas alcaliphila, Pseudomonas chlororaphis, Pseudomonas monteilii, Pseudomonas lini, Pseudomonas alkylphenolica, Pseudomonas nitroreducens, Pseudomonas stutzeri, Ochrobactrum anthropic, Ochrobactrum intermedium, Aeromonas media, Aeromonas veronii, Aeromonas jandaei, Aeromonas hydrophila, Cellulomonas chitinilytica, Cellulomonas cellasea, Cellulomonas hominis, Flavimobis soli, Flavobacterium banpakuense, Buttiauxella agrestis, Buttiauxella noackiae, Achromobacter denitrificans, Pelosinus fermentans, Variovorax dokdonensis, Hydrogenophaga bisanensis, Raoultella terrigena, Delftia lacustris, Shewanella putrefaciens, Acidovorax soli, Hyphomicrobium denitrificans. Microvirgula aerodenitrificans, Candidatus kuenenia, Candidatus brocadia, Candidatus anammoxoglobus, Candidatus jettenia, and Candidatus scalindua.

As can be seen from the Examples, the method of the invention, when applied to drinking water, provides for ammonia removal of more than 1 kg N/m3 d. In one embodiment, the method removes ammonia more than 1 kg N/m3 d. In a further embodiment, the specific activity (mg-N removed per kilogram of biobeads per hour (mg-N/kg·hr)) of the polymer support of immobilized microorganisms is such that the ammonia removal is at least 1 kg N/m3 d. From the Examples, it is seen that the polymer support of immobilized microorganisms demonstrated a sharply increasing rate of TN removal from day 1. In a suitable embodiment of the method of the invention, the TN removal remains consistent, such as from 50-100 mg-N/kg·hr, such as at 70-80 mg-N/kg·hr as demonstrated in the Examples.

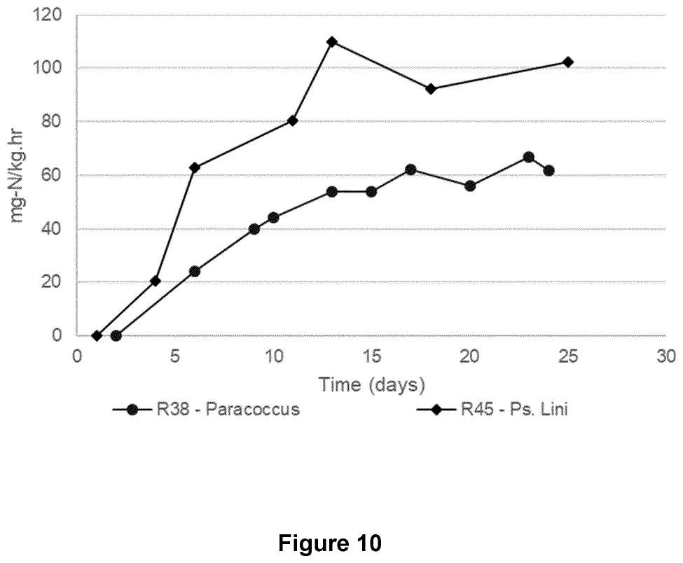

While nitrate removal is the primary objective of drinking water treatment processes, this must be achieved with minimal production of nitrite (NO2) during the process. Accordingly, an embodiment relates to a method for removing nitrate from a drinking water source, while simultaneously removing nitrite. In a typical embodiment, given the MCL for nitrate is 10 mg-N/L and the MCL for nitrite is 1 mg-N/L, the formation of nitrite as a by-product in the nitrate removal process is monitored. The proportion of the NO3 removed that was converted to nitrite and left the reactor in the effluent is illustrated in FIG. 10.

The Examples clearly demonstrated the advantages of applying seeded biobeads in a biological nitrate removal system for drinking water treatment. Under commercially applicable conditions, the seeded biobeads in R38 were able to achieve the goal of an effluent concentration of <2 mg-N/L of total nitrogen (nitrate and nitrite), while limiting the production of nitrite to a level where it is acceptable to meet the MCL in most of the USA.

FISH analysis confirmed that the seeded microbes still dominated the microbial community in the biobeads even after 44 days and 122 days of continuous operation.

The Examples demonstrated that unique microbes with specific commercial advantageous capabilities can be successfully integrated into the PVA-GA biobeads while maintaining the specific advantageous capabilities. In this case, the Pseudomonas lini denitrifying microorganism was integrated into the PVA-GA biobead. The expected advantages of high denitrification activity and significantly lower carbon source consumption were realized. The average 28% lower carbon source consumption while achieving higher rates of denitrification can have significant financial and competitive advantages for operators of biological denitrification systems for drinking water and wastewater treatment that utilize Pseudomonas lini in a PVA-GA biobead.

The level of activity, the level to which the levels of ammonia are to be lowered can, at least in part, be regulated by control of microbial load and/or the bead load. The microbial load is the concentration of microorganisms immobilized within the polymer support. The bead load is the concentration of polymer supports (beads) per unit volume of the water tank, bed, pond or treatment system. In a suitable embodiment, the polymer support is combined with the water at a bead load of 5% to 30 w/v, such as 10% to 30% w/v, as 15% to 30% w/v, such as 15% w/v, 20% w/v, 25% w/v or 30% w/v. For denitrification of water, wherein the microorganisms immobilized within the polymer support are selected from Pseudomonas lini and Paracoccus pantotrophus, and combinations thereof, the microbial load may be selected from 5 g/kg bead to 300 g/kg, typically 10 g/kg to 250 g/kg, such as 10 g/kg, 20 g/kg, 30 g/kg, 40 g/kg, 50 g/kg, 60 g/kg, 70 g/kg, 90 g/kg, 100 g/kg, 110 g/kg, 120 g/kg, 130 g/kg, 140 g/kg, 150 g/kg, 200 g/kg or 250 g/kg.

A related aspect of the invention is directed to a method of reducing the odour of water comprising the use of a polymer support as defined herein. In drinking water, the presence of ammonium (NH₄⁺) will lead to nitrification in the distribution network that can lead to aesthetic issues (taste and odour), corrosion, alkalinity consumption and decreased pH. This uncontrolled nitrification can also lead to incomplete nitrification, resulting in the production of nitrite (NO₂⁻), a toxic intermediate. The presence of ammonium can also increase chlorine demand, which then increases the presence of disinfection by-products and increases the potential for unwanted growth in distribution systems. In areas where drinking water sources are anoxic groundwater resources, the presence of nitrate (NO₃⁻) can be an issue. Nitrate can impact how blood transports oxygen, especially in babies, leading to “blue baby syndrome”.

The goal of drinking water treatment is to produce safe and reliable water for human and industrial consumption. Depending on the source of the water, this may require the removal or reduction in concentration of ammonium or nitrite/nitrate before it can be considered safe for consumption. For one skilled in the art, the process steps for drinking water treatment are well known. Once water is extracted from the source, a primary treatment may be applied to remove large particles or other contaminants including, but not limited to organic matter, iron, manganese. This is generally not required when the source is groundwater.

After any required primary treatment, the water would pass directly to an immobilized biological dentification process. In a suitable example, the water could pass through a short de-oxygenation stage to lower the dissolved oxygen concentration in the water to between 0.0-0.3 mg/L, most preferably to 0.0-0.1 mg/L.

The effluent from the denitrification step should have a NOₓ (NO₂⁻+NO₃⁻) concentration that is between 0.0-5.0 mg/L, more preferably between 0.0-2.0 mg/L and most preferably between 0.0-1.0 mg/L. After denitrification, the water can then be oxygenated to the required level in the aeration ladder, followed by other treatment to ensure the quality requirements for the water met, including filtration to remove solids and disinfection to ensure the safe delivery of the drinking water.

The two-step process of nitrogen bio-elimination from drinking water generally consists of nitrification under strict aerobic conditions followed by denitrification under anoxic conditions. Ammonia primarily present in wastewaters are being oxidized to nitrite and eventually nitrate with the help of obligate aerobic autotrophs known as ammonia-oxidizing bacteria (AOB) such as Nitrosomonas, Nitrosococcus. The NO₂⁻ to NO₃⁻ conversion is fulfilled by nitrite-oxidizing bacteria (NOB) Nitrobacter. Accordingly, the polymer support of immobilized microorganisms may be such that the ammonia oxidizing bacteria are selected from Nitrosomonas spp., Nitrosococcus spp., Nitrosospira spp., Nitrosolobus spp., and Nitrosovibrio spp, and wherein the nitrite oxidizing bacteria are selected from Nitrobacter spp., Nitrococcus spp., Nitrospira spp., and Nitrospina spp. In a preferred embodiment, the microorganisms are a combination of the ammonia oxidizing bacteria Nitrosomas spp. and the nitrite oxidizing microorganisms are Nitrobacter spp. In an embodiment of the invention, the microorganisms are selected from the group consisting of a combination of Nitrosomonas eutropha and a Nitrobacter, and a combination of a Nitrosomonas europaea and a Nitrobacter. In a related embodiment, the microorganisms are a combination of a Nitrosomonas and Nitrobacter winogradsky, such as wherein the microorganisms are selected from the group consisting of a combination of Nitrosomonas eutropha and Nitrobacter winogradskyi, and a combination of Nitrosomonas europaea and Nitrobacter winogradskyi, preferably a combination of Nitrosomonas eutropha and Nitrobacter winogradskyi.

Denitrification occurs under anoxic/anaerobic conditions. Denitrification is the sequential process involving the dissimilatory reduction of one or both the ionic nitrogen oxides, nitrate (NO₃⁻) and nitrite (NO₂⁻) to gaseous nitrogen oxides, nitric oxide (NO), nitrous oxide (N₂O) and finally reduce to the ultimate product, dinitrogen (N₂) thus removing biologically available nitrogen and returning it to the atmosphere.

Both nitrate and nitrite are fully converted to atmospheric nitrogen. However, insufficient carbon sources, low dissolved oxygen (DO) concentrations and operational fluctuations or environmental conditions lead to improper denitrification and N₂O accumulation and emissions. One pathway to reduce N₂O production is to select the right organisms for the denitrification process.

Denitrifiers with their facultative anaerobic traits perform denitrifying activities under the presence of oxygen driving an increase in N₂O as a denitrification intermediate. Many heterotrophic nitrifiers along with the oxidation of NH₃ can simultaneously perform aerobic denitrification. N₂O is then generated. Accordingly, the judicious selection of denitrifiers is an important aspect of the present invention.

Suitable embodiment of microorganism for this aspect of the invention may be selected from the group consisting of Pseudomonas spp; Paracoccus spp, Janthinobacterium, Microvirgula aerodenitrificans and Castellaniella defragrans, particularly Pseudomonas lini and Paracoccus pantotrophus,

The oxidation of the ammonium to nitrogen gas may be achieved in wastewater treatment processes using the polymer carrier of the invention. Suitably, the two step conversion comprise the autotrophic organisms, Nitrosomonas and Nitrobacter, and many different heterotrophs. The former obtain energy from the oxidation of ammonia, obtain carbon from CO₂, and use oxygen as the electron acceptor. They are termed autotrophic because of their carbon source and termed aerobes because of their aerobic environment. The heterotrophic organisms are responsible for denitrification or the reduction of nitrate, NO₃⁻, to nitrogen gas, N₂. They use carbon from complex organic compounds, prefer low to zero dissolved oxygen, and use nitrate as the electron acceptor.

According to the present invention simultaneous nitrification-denitrification may be achieved by immobilizing both autotrophic bacteria and heterotrophic bacteria in one polymer hydrogel support or by immobilizing an autotroph in one polymer support and a heterotroph in a second polymer hydrogel support, with strict control of dissolved oxygen.

An embodiment of the method of the invention for nitrification involves developing an oxygen gradient by adding oxygen in one location in the basin. Near the O₂ injection point, a high DO concentration is maintained allowing for nitrification and oxidation of other organic compounds. Oxygen is the electron acceptor and is depleted. The DO level in localized environments decreases with increasing distance from the injection point. In these low DO locations, the heterotrophic bacteria complete the nitrogen removal.

Another embodiment comprises establishing an oxygen gradient within the polymer beads that immobilize the microorganisms. The DO concentration remains high in the outside rings of the beads where nitrification occurs but low in the inner rings of the beads where denitrification occurs.

In this aspect of the invention, a single denitrifying strain, such as a Paracoccus, in the bead creates an oxygen gradient with an aerobic and anoxic environment allowing for both nitrification and denitrification. In a typical embodiment, the outer portion of the polymer support has access to oxygen, thus allowing for an aerobic process, and the oxygen of aerobic medium/environment is consumed prior to the medium/environment enters the interior portion of the polymer support wherein an anoxic process is performed.

Typically, simultaneous nitrification and denitrification (SNdN) has slower ammonia and nitrate utilization rates as compared to separate basin designs because only a fraction of the total biomass is participating in either the nitrification or the denitrification steps. Another embodiment comprises autotrophic denitrifying bacteria in the process termed the Anammox process. In one embodiment, the microorganism is selected from the group consisting of Microvirgular aerodenitrificans, Paracoccus pantotrophus, Castellaniella defragrans and Pseudomonas lini, particularly Pseudomonas lini or Paracoccus pantotrophus,

The microorganisms may be a combination of ammonium oxidizing microorganisms and nitrite oxidizing microorganisms. The microorganisms may be a combination of the ammonia oxidizing bacteria Nitrosomas spp. and the nitrite oxidizing microorganisms Nitrobacter spp. The microorganisms are selected from the group consisting of a combination of Nitrosomonas eutropha and a Nitrobacter, and a combination of a Nitrosomonas europaea and a Nitrobacter. The microorganisms may be a combination of a Nitrosomonas and Nitrobacter winogradsky. The microorganisms are selected from the group consisting of a combination of Nitrosomonas eutropha and Nitrobacter winogradskyi, and a combination of Nitrosomonas europaea and Nitrobacter winogradskyi, preferably a combination of Nitrosomonas eutropha and Nitrobacter winogradskyi. The microorganisms may be wherein the ammonia oxidizing bacteria are selected from Nitrosomonas spp., Nitrosococcus spp., Nitrosospira spp., Nitrosolobus spp., and Nitrosovibrio spp, and wherein the nitrite oxidizing bacteria are selected from Nitrobacter spp., Nitrococcus spp., Nitrospira spp., and Nitrospina spp.

In a further embodiment, a denitrifying bacteria partially nitrifies the ammonia, that is to say from ammonia to nitrite. In this embodiment of the process, the nitrite is then leaked out of the polymer support. This process is used in combination with the annamox process, that is depended on a supply of nitrite.

The AOB may belong to the species Nitrosomonas eutropha and/or it may have a 16S rDNA sequence which is less than 2% dissimilar from (more than 98% identical to) SEQ ID NO: 1 disclosed in WO2006044499A2, particularly less than 1% dissimilar (more than 99% identical). Preferably, the AOB has a 16S rDNA sequence which is SEQ ID NO: 1 disclosed in WO2006044499A2 or is the Nitrosomonas eutropha strain contained in ATCC PTA-6232.

The NOB may belong to Nitrobacter winogradskyi and/or it may have a 16S rDNA sequence which is less than 10% dissimilar from (more than 90% identical to) SEQ ID NO: 2 disclosed in WO2006044499A2, particularly less than 6% or less than 3% dissimilar (more than 94% or more than 97% identical). Preferably, the NOB has a 16S rDNA sequence which is SEQ ID NO: 2 or is the Nitrobacter winogradskyi strain contained in ATCC PTA-6232. A given sequence may be aligned with SEQ ID NO: 1 or 2 and the dissimilarity or identity may be calculated using the BLAST program (Basic Local Alignment Search Tool, available at www.ebi.ac.uk/blast/index.html where the expectation value is set at 10, the penalty for nucleotide mismatch is −3, the reward for match is +1, the gap opening penalty is −5 and the gap extension penalty is −2. A sequence alignment may be produced using the CLUSTALW program from the PHYLIP Phylogenetic Inference Package (Felsenstein, J. 1989. PHYLIP—Phylogeny Inference Package (Version 3.2). Cladistics 5: 164-166). The Accurate Method using the IUB/BESTFIT weight matrix may be used with a gap penalty of −15 and an extension penalty of −6.66. The resulting alignment may be used to determine % dissimilarity (and % identity) using the DNADIST program from PHYLIP according to the Jukes-Cantor model.

The AOB or NOB may be combined with other bacteria, e.g., Bacillus such as a combination of the commercial product Prawn Bac PB-628 (product of Novozymes Biologicals), together with Enterobacter or Pseudomonas. The nitrifying consortium may be formulated as a liquid, a lyophilized powder, or a biofilm, e.g., on bran or corn gluten. The ammonia oxidizing bacterium will typically be inoculated to an ammonia oxidation rate of about 50-5.000 mg NH3-N/L/hr (typically around 800), and the nitrite oxidizing bacterium will typically be inoculated to a nitrite oxidizing rate of about 10-2.000 mg NO₂⁻—N/L/hr (typically around 275).

Anammox is the oxidation of ammonium with nitrite as the electron acceptor and dinitrogen gas as the product. Another embodiment comprises autotrophic denitrifying bacteria in the process termed the Anammox process. The process may be mediated by obligately anaerobic chemolithoautotrophic bacteria that form a monophyletic cluster inside the Planctomycetales, one of the major divisions of the bacteria. The anammox bacteria may be selected from the group consisting of C. brocadia anammoxidans, Candidatus kuenenia stuttgartiensis, Candidatus scalindua wagneri, and Candidatus scalindua brodae.

Typically, SNdN has slower ammonia and nitrate utilization rates as compared to separate basin designs because only a fraction of the total biomass is participating in either the nitrification or the denitrification steps.

The efficacy of biological nitrogen removal processes is herein enhanced through the application of high concentrations of nitrifying/denitrifying organisms in a biological treatment process. In order to reduce or eliminate the production of biomass in the effluent from the process, according to the invention, selected microorganisms are immobilized.

One aspect of the invention is related to the use of the polymer support of immobilized microorganisms for reducing the levels or removal of nitrogen containing compounds selected from the group consisting of ammonia, nitrites or nitrates from wastewater. In an embodiment, the reduction in levels of ammonia comprises said use wherein immobilized microorganisms are ammonia-oxidizing bacteria (AOB). In an embodiment, the reduction in levels of nitrites comprises said use wherein immobilized microorganisms are nitrite-oxidizing bacteria (NOB). In an embodiment, the reduction in levels of nitrates comprises said use wherein immobilized microorganisms are heterotrophic bacteria.

A related aspect of the invention is directed to a method of treating wastewater for nitrification of ammonium to nitrate, the dentification of nitrate, and nitrate to nitrogen, or combinations thereof comprising the addition of the polymer support of immobilized microorganisms

In a typical embodiment, the process of nitrogen bio-elimination from wastewater comprises a nitrification step under strict aerobic conditions using ammonia-oxidizing bacteria (AOB) followed by a denitrification step under anoxic conditions using nitrite-oxidizing bacteria (NOB).

When considering the application of polymer support comprising immobilized microorganisms for nitrogen treatment, the requirements will depend on the design of the site. The polymer support comprising immobilized microorganisms are suitably for tertiary treatment, side-stream treatment and integrated immobilized microbe activated sludge (IIMAS) processes.

A further aspect is directed to the use of the immobilized microorganisms of the invention for tertiary nitrification and/or denitrification treatment in a wastewater treatment process. In a suitable embodiment, raw wastewater passes through primary clarification where large solids, fats and grit are removed from the wastewater. Chemical precipitants may be added at this stage to remove soluble phosphorus from the wastewater. In the next step, the immobilized microorganisms are combined with the primary treated water. In this embodiment of the process, activated sludge may be utilised to encourage the removal of nutrients, including but not limited to, nitrogen, phosphorus and biochemical oxygen demand (BOD). In a typical embodiment, ammonium is nitrified to nitrate.

In a preferred embodiment, the method of the invention removes ammonium and nitrite and nitrate (NOₓ) is removed or reduced in a combined nitrification/denitrification process. The microbial biomass can be in the form of activated sludge or a fixed biofilm on a carrier or bearing material.

The mixed liquor (wastewater and biomass) is then typically passed to the secondary clarifier. In this embodiment the biomass solids settle under gravity and are separated from the treated water. In a preferable embodiment, a membrane is used to separate the biomass from the treated wastewater. A proportion of the biomass produced in the biological wastewater treatment process is then returned to the biological treatment process, with the remainder removed from the process as a by-product.

In this embodiment, the effluent from the secondary clarifier may not necessarily meet the requirement for environmental discharge. Therefore, a tertiary nitrogen treatment process is typically required. This process would, according to the invention, use immobilized microorganisms in a nitrification and/or denitrification process to produce an effluent that meets the required standards.

In a further embodiment, the immobilized microbial nitrification and/or denitrification process may be utilized to treat a high strength side-stream of wastewater before it is blended into the primary treated wastewater. Examples of such side-stream wastewaters can be, but not limited to, the liquid fraction from anaerobic digestate dewatering processes, industrial wastewater effluents and septic tanks. The high strength wastewater, if it meets certain quality standards including, but not limited to, total suspended solids (TSS) concentration, BOD concentration, pH, temperature, may be treated according to the invention for the nitrification and/or denitrification of the side-stream wastewater. The application of a compact, high rate biological process such as the immobilized microorganisms, advantageously reduces the ammonium, nitrate or nitrite loading that is applied to the biological treatment process. This can ensure the process is not overloaded and can continue to meet its obligations with regards to effluent quality. The advantages of the application of an immobilized microorganism process on side-stream wastewater treatment include, but not are not limited to, a resistance to operational conditions such as pH, temperature and detrimental components in the wastewater and the process does not produce solids that could create issues for the following biological treatment process. The application of a side-stream immobilized treatment process does not absolutely remove the requirement for a tertiary immobilized microorganism process as outlined earlier.

In a further embodiment, the immobilized microorganisms may be applied directly in the biological treatment process. This application is similar to an Integrated Fixed-film Activated Sludge (IFAS) system. However, instead of using fixed film carriers to provide a means of retaining biomass in the system, immobilized microorganisms are used to boost the amount of microorganisms with a specific activity required by the process, for example by integrating immobilized nitrifying microorganisms to specifically boost the nitrification activity in the process. These immobilized microorganisms also have the advantage of being able to be easily retained in the activated sludge process through physical separation techniques that would be well known to one skilled in the art. This process could be considered as being an Integrated Immobilized Microorganism Activated Sludge (IIMAS) process.

In an IIMAS process, the immobilized microorganisms are applied directly into the former biological treatment process. This process is most preferably an activated sludge process. The immobilized microorganisms supplement the activity of the biological treatment process through the supplementation of the already existing biomass with high activity microorganisms for nitrification and/or denitrification.

The advantages of an IIMAS type application include, but are not limited to, an increased resistance of the process to operational shocks including pH, temperature and the concentration of components in the wastewater. The immobilized microorganisms would add nitrification and/or denitrification activity to the activated sludge process without increasing the production of biomass, thereby saving on operational costs. The application of an IIMAS type system has the potential to increase volumetric treatment capacity of the biological treatment process, thereby allowing the biological treatment process to treat more wastewater without increasing the volume of the treatment basins.

During the treatment of the water according to the invention, the polymer support retains the microorganism but leakage of the microorganism through the pores and out of the polymer and into the treatment basins during the nitrification and denitrification steps of nitrogen removal.

It has been demonstrated (see Example 10) that the polymer support of the invention are able to provide a protective environment for microbes that have been integrated as part of the production process of the biobeads. While microbes are able to colonize the outer surfaces of blank (not comprising immobilized microorganisms or not seeded) polymer support, these microbes are not protected against exposure to toxins or inhibitors, contrary to the immobilized microorganisms of the invention that are within the biobeads.

This property of the immobilized microorganisms with the polymer support is highly advantageous in that biological nitrogen removal processes are often exposed to toxins and inhibitors, with chlorine being one of the most effective microbial contamination control chemicals used, with chlorine used to ensure the disinfection of drinking water by killing any suspended microbes in the water. Advantageously, biological nitrogen removal process utilizing the polymer support with immobilized microorganisms of the invention are resistant to the impact of toxins such as chlorine exposure and furthermore recover its nitrogen removal activity in a very short period of time. This proceeds significant value for operators using a the polymer support with immobilized microorganisms of the invention as the polymer support ensures that exposure to toxins has a minimal impact on TN removal compared to a comparable suspended or fixed film system.

### PREFERRED EMBODIMENTS

1. A polymer support of immobilized microorganisms
a. wherein the polymer support comprises said microorganisms immobilized within a polymer hydrogel;
b. wherein the polymer hydrogel comprises
   i. polyvinyl alcohol polymeric chains cross linked with glutaraldehyde; and
   ii. water or an aqueous solution.

2. The polymer support of immobilized microorganisms according to embodiment 1 wherein a covalent bond between glutaraldehyde and polyvinyl alcohol polymeric chains crosslinks the polyvinyl alcohol polymeric chains.

3. The polymer support of immobilized microorganisms according to embodiments 1 or 2 wherein the polyvinyl alcohol polymeric chains consist of 500-3.000 monomer units and a molecular weight of 22.000 g/mol to 130.000 g/mol.

4. The polymer support of immobilized microorganisms according to embodiments 1 to 3, wherein the crosslink comprises monomeric, dimeric, oligomeric, or polymeric glutaraldehyde.

5. The polymer support of immobilized microorganisms according to embodiments 1 to 4, wherein the crosslinking density, determined as the number of glutaraldehyde crosslinking units, is 8 to 25%, such as 10 to 20%, such as 12-20%, such as about 12%, 13%, 15%, 16%, 17%, 18%, 19% or 20%.

6. The polymer support of immobilized microorganisms according to embodiments 1 or 5, wherein polymer hydrogel comprises from 60 to 99 wt % cross-linked polymeric material and 1 to 40 wt % water or aqueous medium, such as from 60 to 98 wt % cross-linked polymeric material and 2 to 40 wt % water or aqueous medium, such as from 65 to 95 wt % cross-linked polymeric material and 5 to 35 wt % water or aqueous medium, such as from 65 to 90 wt % cross-linked polymeric material and 10 to 35 wt % water or aqueous medium, such as from 65 to 85 wt % cross-linked polymeric material and 15 to 35 wt % water or aqueous medium, such as from 65 to 80 wt % cross-linked polymeric material and 20 to 35 wt % water or aqueous medium, or such as from 65 to 75 wt % cross-linked polymeric material and 25 to 35 wt % water or aqueous medium.

7. The polymer support of immobilized microorganisms according to embodiments 1 or 6 further comprising alginate in monomer or polymeric form, entangled with cross-linked polyvinyl alcohol polymeric chains so as to form an interpenetrating polymer network.

8. A polymer support according to embodiments 1 to 7, wherein the microorganisms are selected from the group consisting of ammonium oxidizing microorganisms, nitrite oxidizing microorganisms, denitrifying microorganisms, combinations thereof and anammox bacteria.

9. The polymer support of immobilized microorganisms according to embodiments 1 to 7, wherein the microorganisms are selected from a mixed or pure culture of nitrite-oxidizing bacteria, a mixed or pure culture of ammonium oxidizing bacteria, a mixed or pure culture of ammonium oxidizing and nitrite-oxidizing bacteria, a mixed or pure culture of denitrifying bacteria and a mixed or pure culture of anammox bacteria.

10. The polymer support of immobilized microorganisms according to any of the preceding embodiments, wherein the microorganisms are a combination of ammonium oxidizing microorganisms and nitrite oxidizing microorganisms.

11. The polymer support of immobilized microorganisms according to any of the preceding embodiments, wherein the microorganisms are a combination of the ammonia oxidizing bacteria Nitrosomonas spp. and the nitrite oxidizing microorganisms Nitrobacter spp.

12. The polymer support of immobilized microorganisms according to any of the preceding embodiments, wherein the microorganisms are selected from the group consisting of a combination of Nitrosomonas eutropha and a Nitrobacter, and a combination of a Nitrosomonas europaea and a Nitrobacter.

13. The polymer support of immobilized microorganisms according to any of the preceding embodiments, wherein the microorganisms are a combination of a Nitrosomonas and Nitrobacter winogradskyi.

14. The polymer support of immobilized microorganisms according to any of the preceding embodiments, wherein the microorganisms are selected from the group consisting of a combination of Nitrosomonas eutropha and Nitrobacter winogradskyi, and a combination of Nitrosomonas europaea and Nitrobacter winogradskyi, preferably a combination of Nitrosomonas eutropha and Nitrobacter winogradskyi.

15. The polymer support of immobilized microorganisms according to any of the preceding embodiments, wherein the ammonia oxidizing bacteria are selected from Nitrosomonas spp., Nitrosococcus spp., Nitrosospira spp., Nitrosolobus spp., and Nitrosovibrio spp, and wherein the nitrite oxidizing bacteria are selected from Nitrobacter spp., Nitrococcus spp., Nitrospira spp., and Nitrospina spp.

16. The polymer support of immobilized microorganisms according to any of the preceding embodiments, wherein the microorganisms immobilized within the polymer hydrogel are selected from the group consisting of Pseudomonas lini, Paracoccus pantotrophus, Paracoccus pandtodrophus, Castellaniella defragans, Pseudomonas proteolytica, Paracoccus versutus, Paracoccus denitrificans, Nitrosomonas eutropha, Nitrosomonas europaea, and Nitrobacter winogradskyi,

17. The polymer support of immobilized microorganisms according to any of the preceding embodiments, wherein the immobilized microorgansims comprise Pseudomonas lini.

18. The polymer support of immobilized microorganisms according to any of the preceding embodiments, wherein the immobilized microorgansims comprise Paracoccus pantotrophus.

19. The polymer support of immobilized microorganisms according to any of the preceding embodiments, wherein the immobilized microorgansims comprise Castellaniella defragans.

20. The polymer support of immobilized microorganisms according to any of the preceding embodiments, wherein the immobilized microorgansims comprise Pseudomonas proteolytica.

21. The polymer support of immobilized microorganisms according to any of the preceding embodiments, wherein the immobilized microorgansims comprise Paracoccus versutus.

22. The polymer support of immobilized microorganisms according to any of the preceding embodiments, wherein the immobilized microorgansims comprise Paracoccus denitrificans.

23. The polymer support of immobilized microorganisms according to any of the preceding embodiments, wherein the immobilized microorgansims comprise Nitrosomonas eutropha.

24. The polymer support of immobilized microorganisms according to any of the preceding embodiments, wherein the immobilized microorgansims comprise Nitrosomonas europaea.

25. The polymer support of immobilized microorganisms according to any of the preceding embodiments, wherein the immobilized microorgansims comprise Nitrobacter winogradsky.

26. The polymer support of immobilized microorganisms according to any of the preceding embodiments, wherein the polymer support is porous.

27. The polymer support of immobilized microorganisms according to embodiment 26, wherein the polymer support comprises micropores.

28. The polymer support of immobilized microorganisms according to any of embodiments 26 and 27, wherein the polymer support comprises macropores.

29. The polymer support of immobilized microorganisms according to any of embodiments 26 to 28, comprising one or more macropores in the central volume of support and micropores.

30. The polymer support of immobilized microorganisms according to any of embodiments 26 to 29, wherein a portion of the polymer support comprises micropores with an average pore diameter of 5 to 40 microns, such as 5 to 30 microns, such as 5 to 20 microns.

31. The polymer support of immobilized microorganisms according to any of embodiments 26 to 30, wherein a portion of the polymer support comprises micropores with an average pore diameter of 10 to 40 microns, such as 20 to 40 microns.

32. The polymer support of immobilized microorganisms according to any of the preceding claims wherein the polymeric support is spherical, oval, elliptical bead-shaped, oblong, cylindrical, or capsule-like in shape.

33. The polymer support of immobilized microorganisms according to any of the preceding claims wherein the polymeric support is spherical or bead-shaped having a diameter of 1 to 10 mm, typically 2 to 8 mm, such as 3 to 7 mm or 3 to 6 mm.

34. The polymer support of immobilized microorganisms according to any of the preceding claims, comprising a microbial load of 5 g/kg bead to 250 g/kg, typically 10 g/kg to 150 g/kg.

35. A method of treating water, such as any of drinking water, municipal wastewater or industrial wastewater, such as any of drinking water or municipal wastewater comprising mixing said water a polymer support as defined in any of embodiments 1 to 34.

36. A method according to embodiment 35, wherein the polymer support is combined with the water at a bead load of 5% to 30 w/v, such as 10% to 30% w/v, as 15% to 30% w/v.

37. A method according to any of embodiments 35 to 26, wherein the wastewater comprises from 5 to 400 mg/L of ammonia, such as 5 to 200 mg/L ammonia, such as 5 to 100 mg/L of ammonia, such as 10 to 80 mg/l, such as 15 to 60, of ammonia.

38. A method of reducing the amount of ammonia in wastewater comprising adding to the wastewater a polymer support as defined in any of embodiments 1 to 34, wherein the microorganism is selected from Nitrosomonas eutropha, Nitrobacter winogradskyi and combinations thereof.

39. The method according to embodiment 38, wherein the polymer support is combined with the water at a bead load of 5% to 30 w/v, such as 10% to 30% w/v, as 15% to 30% w/v.

40. The method according to any of embodiments 38 to 39, wherein the Nitrosomonas eutropha, Nitrobacter winogradskyi and combinations thereof are at a microbial load of 5 g/kg bead to 250 g/kg, typically 10 g/kg to 150 g/kg.

41. A method of denitrifying water comprising adding to the wastewater a polymer support as defined in any of embodiments 1 to 34, wherein the microorganism is selected from the group consisting of Pseudomonas lini, Paracoccus pantotrophus and combinations thereof.

42. A method of reducing the odour of water comprising the use of a polymer support as defined in any of embodiments 1 to 34.

43. A method of preparing a polymer support of immobilized microorganisms, said method comprising
1a. Combining in solution a source of alginate, polyvinyl alcohol, and microorganisms into a mixture;
1b. Adding said mixture to a Ca²⁺-containing solution forming a heterogenous solution comprising a gelate structure;
2a. Adding said gelate structure to a cross-linking solution, said cross-linking solution comprising glutaraldehyde;
2b. Mixing said gelate structure in said cross-linking solution so as to form a polymer support of immobilized microorganisms.

### EXAMPLES

### Materials and Methods

Mowiol® is a commercially available water-soluble hydrocolloid based on poly (vinyl alcohol) (PVA). Poval® 15-99) is a commercially available water-soluble hydrocolloid based on poly (vinyl alcohol) (PVA)

Nitrosomonas eutropha and Nitrobacter winogradskyi are commercially available and produced by Novozymes as Prawnbac® NNC.

TABLE 1
List of components analysed in the feed and
effluent water from the reactors

| Compound | Name | Unit of measurement |
|---|---|---|
| NO₃ | Nitrate | mg-N/L |
| NO₂ | Nitrite | mg-N/L |
| NH₄ | Ammonia | mg-N/L |
| TN | Total Nitrogen | mg-N/L |
| PO₄ | Ortho-phosphate | mg-P/L |
| SCOD | Soluble chemical oxygen | mg-O₂/L |
| | demand | |
| TSS | Total suspended solids | mg/L |

### Example 1—Paracoccus Containing PVA-GA-Beads

13 g PVA (Poval 15-99) was filled up with 87 g tap water and autoclaved for 1 h to dissolve the PVA. Then the solution was cooled to room temperature. Additionally, an 8% Sodium Alginate (Satialgine S60NS) solution is prepared.

40 g of the PVA solution, 8 g of the Alginate solution and 16 g of a “Paracoccus” microbe suspension (50 g centrifuged cells per L) is mixed and subsequently dropped into a 1% CaCl₂) solution via a peristaltic pump with a pumping speed of 200 g/h. The outlet of the tubes coming from the peristaltic pump have a inner diameter of 1.8 mm and the tips were positioned 5-10 cm from the liquid surface.

The dropped polymer/microbe mixture immediately gelate when hitting the CaCl₂) bath. After adding 50 g of the mix into 50 g of the CaCl₂) bath, the spherical products were separated using a small sieve and washed lightly with tap water before transferring the preformed product back into a glass beaker.

On top of these spheres a second cross link solution is added containing 0.25 g glutaraldehyde (10% solution), 1.7 g sulfuric acid (30% solution), 10 g of Na₂SO₄ and 38.05 g water. This second cross link solution is heated to 40° C. and is kept at this temperature during Bead cross linking. After 3 hours of curing the beads are separated from the cross-link solution and washed with a tris buffer for 30 min. After washing the Beads are transferred into cell free water for storage.

### Example 2—Nitrosomonas eutropha and Nitrobacter Winogradskyi-Containing PVA-GA-Beads

13 g PVA (Poval 15-99) was filled up with 87 g tap water and autoclaved for 1 h to dissolve the PVA. Then the solution was cooled to room temperature. Additionally, an 8% Sodium Alginate (Satialgine S60NS) solution is prepared.

40 g of the PVA solution, 8 g of the Alginate solution and 16 g of a “Prawnbac” microbe suspension (500 g centrifuged cells per L) is mixed and subsequently dropped into a 1% CaCl2 solution via a peristaltic pump with a pumping speed of 200 g/h. The outlet of the tubes coming from the peristaltic pump have an inner diameter of 1.8 mm and the tips were positioned 5-10 cm from the liquid surface.

The dropped polymer/microbe mixture immediately gelate when hitting the CaCl₂ bath. After adding 50 g of the mix into 50 g of the CaCl₂) bath, the spherical products were separated using a small sieve and washed lightly with tap water before transferring the preformed product back into a glass beaker.

On top of these spheres a second cross link solution is added containing 1 g glutaraldehyde (10% solution), 1.7 g sulfuric acid (30% solution), 10 g of Na₂SO₄ and 37.3 g water. This second cross link solution is heated to 40° C. and is kept at this temperature during Bead cross linking. After 3 hours of curing the beads are separated from the cross-link solution and washed with a tris buffer for 30 min. After washing the Beads are transferred into cell free water for storage.

### Example 3—Nitrosomonas eutropha and Nitrobacter winogradskyi-Containing PVA-GA-Beads

13 g PVA (Poval 15-99) was filled up with 87 g tap water and autoclaved for 1 h to dissolve the PVA. Then the solution was cooled to room temperature. Additionally, an 8% Sodium Alginate (Satialgine S60NS) solution is prepared.

40 g of the PVA solution, 8 g of the Alginate solution and 16 g of a diluted “Prawnback” microbe suspension (500 g centrifuged cells per L are diluted 1:4 with water to 125 g/L) is mixed and subsequently dropped into a 1% CaCl₂) solution via a peristaltic pump with a pumping speed of 200 g/h. The outlet of the tubes coming from the peristaltic pump have a inner diameter of 1.8 mm and the tips were positioned 5-10 cm from the liquid surface.

The dropped polymer/microbe mixture immediately gelate when hitting the CaCl2 bath. After adding 50 g of the mix into 50 g of the CaCl₂) bath, the spherical products were separated using a small sieve and washed lightly with tap water before transferring the preformed product back into a glass beaker.

On top of these spheres a second cross link solution is added containing 0.25 g glutaraldehyde (10% solution), 1.7 g sulfuric acid (30% solution), 10 g of Na₂SO₄ and 38.05 g water. This second cross link solution is heated to 40° C. and is kept at this temperature during Bead cross linking. After 3 hours of curing the beads are sepa-rated from the cross-link solution and washed with a tris buffer for 30 min. After washing the Beads are trans-ferred into cell free water for storage.

### Example 4—Paracoccus pantotrophus-Containing PVA-GA-Beads

The conditions of Example 1 were repeated using Paracoccus pantotrophus,

### Example 5—Demonstration of the Efficacy of Biobeads for Total Nitrogen (TN) Removal from Water at Low Hydraulic Retention Time (HRT) Conditions

### BACKGROUND

There is a growing concern with increasing concentrations of nitrate in drinking water resources in the USA. While there are effective treatment technologies available, these produce a by-product (brine) that is difficult to treat and expensive to dispose of. Therefore, an efficient and effective biological nitrate removal process would be commercially attractive as it would increase the water recovery rate, would not produce a brine and would directly remove the nitrate from the water, rather than simply up-concentrating it in a brine and displacing the nitrate treatment problem to another side. In many areas in the USA, the minimum consent limit (MCL) for nitrate in drinking water is 10 mg-N/L. A publicly available webinar available online from the Arizona Water Association (https://cdn.ymaws.com/www.azwater.org/resource/group/25c2bfe3-42d4-4cd3-9149-4911c8416e5e/Downloads/2017-11-02 Webinar/Nitrate Removal.pdf) introduces the results from pilot trials of a biological nitrate removal process where final nitrate concentrations of <2 mg-N/L were achieved.

First order kinetics in a continuously stirred reactor with a low retention time imply that it is more difficult to achieve high levels of contaminant removal (i.e. very low effluent concentrations) at low feed concentrations. Therefore, in order to challenge the biobeads produced, it was decided to design an experiment with an influent nitrate concentration of 10 mg-N/L (the MCL limit for many jurisdictions in the USA) and a hydraulic retention time of 30 minutes with the goal of achieving a consistent effluent concentration of <2 mg-N/L as nitrate and nitrate (total nitrogen TN).

### Experimental Aims:

Demonstrate the efficacy of biobeads seeded with a known denitrifying micro-organism (Paracoccus pantotrophus) to achieve an effluent concentration of Total Nitrogen (TN)<2.0 mg-N/L at an operational hydraulic retention time (HRT) of 30 minutes

Demonstrate that biobeads seeded with the known denitrifying micro-organism have superior TN treatment capabilities and lower nitrite production compared to biobeads that have not been seeded with the denitrifying microbes during production

Compare the specific biobead activity (mg-N removed per kg biobeads per hour) and effluent quality (mg-N/L) to a theoretical requirement for the biological treatment of nitrate in groundwater based on the example from the Arizona Water Association webinar.

### Methods:

Biobead preparation: The biobeads are prepared as described in Example 4:
Blank biobeads (not comprising the microorganisms)
Seeded biobeads (comprising the microorgansims)

### Lab Scale Reactor Operation

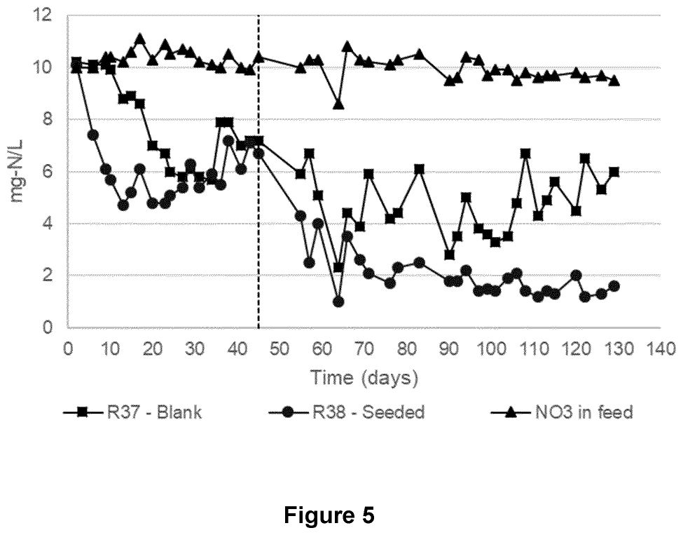

Two reactors were operated under identical conditions for a period of 129 days
Referring to FIG. 5, R37 refers to sample with blank biobeads whereas R38, refers to samples with seeded biobeads.

### General Operational Conditions at the Start of the Trials are Outlined in Table 2

Tap water was used in the experiments
Nitrate was supplied as NaNO₃
BOD was supplied as a 50:50 (on a BOD basis) mixture of sodium acetate and acetic acid
P was supplied as KH₂PO₄
pH was adjusted as required by addition of 10% HCl
A trace elements solution was applied at a concentration of 60 mg/L

TABLE 2
Operational conditions for the reactors from Day 1-45

| Biobead | Reactor conditions | | | | | Substrate characteristics | | |
|---|---|---|---|---|---|---|---|---|
| loading | Volume | Temperature | | HRT | DO | NO₃ | BOD | P |
| (% w/v) | (L) | (deg C) | ph | (h) | (mg/L) | (mg-N/L) | (mg/L) | (mg-P/L) |
| 10 | 0.5 | 15 | 7-8 | 0.5 | 0-1 | 10 | 50 | 2.5 |

The operational conditions were adjusted after 45 days of continual operation as shown in Table 3

TABLE 3
Operational conditions for the reactors from Day 46-129

| Biobead | Reactor conditions | | | | | Substrate characteristics | | |
|---|---|---|---|---|---|---|---|---|
| loading | Volume | Temperature | | HRT | DO | NO₃ | BOD | P |
| (% w/v) | (L) | (deg C) | ph | (h) | (mg/L) | (mg-N/L) | (mg/L) | (mg-P/L) |
| 20 | 0.5 | 15 | 7-8 | 0.5 | 0-1 | 10 | 50 | 2.5 |

### Analytical Methods

Sampling was undertaken three (3) times per week by a certified analytical laboratory

### Results and Discussion:

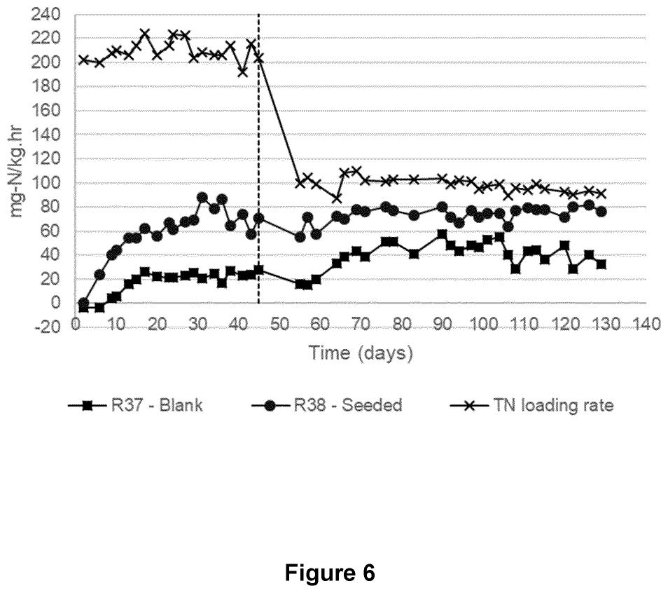

The concentration of nitrate and nitrite in the influent (feed) and effluent from the reactors was measured frequently throughout the trial period. FIG. 6 illustrates the concentration of total nitrogen (nitrate+nitrite) in both the influent and effluent of both R37 and R38. The dotted line at day 45 indicates where the biobead loading (% w/v) was increased from 10% to 20%. The seeded biobeads demonstrated an ability to remove TN immediately, while it took the blank beads at least 10 days to demonstrate any TN removal activity. However, after 27 days of operation, both the blank and seeded biobead reactors were achieving similar results. In both reactors, the effluent concentration was approximately 6 mg-N/L, which was at least 3× higher than required. At day 45, more biobeads were added to each reactor to increase the bead load from 10% to 20% w/v. This increase in bead load in the reactors led to an immediate drop in the effluent concentration of TN, most markedly in the seeded biobead reactor (R38). By day 64, the seeded biobead reactor (R38) had achieved an effluent concentration of <2 mg-N/L, and has largely maintained this level for the following 65 days of operation.

This decrease in TN concentration achieved in R38 after the increase in biobead load in the reactor is due to the increase in active denitrifying microbes in the reactor due to the addition of the extra biobeads. This then allows the biobeads to overcome the limitations of first order kinetics in the continuously stirred reactor and remove more TN from the system. This positive effect of adding more active denitrifying microbes in the biobeads to the reactor is highlighted by the fact that the reactor with blank biobeads (R37) has never demonstrated an effluent concentration of <2 mg-N/L. After an initial sharp decrease in effluent TN concentration after day 45, R37 has maintained an effluent TN concentration that is generally 2× higher than the effluent TN concentration achieved by the seeded biobeads in R38.

The specific activity (mg-N removed per kilogram of biobeads per hour (mg-N/kg·hr)) of the biobeads closely reflects the effluent TN concentrations achieved and illustrated in FIG. 5.

The specific TN loading rate and biobeads activities for R37 and R38 are illustrated in FIG. 6. Again, it is clear that the seeded biobeads in R38 demonstrated a sharply increasing rate of TN removal from day 1. This rate of TN removal stabilized after approximately 20 days and has remained consistent at 70-80 mg-N/kg·hr from day 31 to day 129. The increase in biobead load in the reactor did not impact the specific activity of the biobeads in R38 as the amount of TN removed in the reactor increased sharply with the addition of the extra biobeads (as shown in FIG. 5). The blank biobeads in R37 did not demonstrate significantly activity until approximately day 20, and then remained steady at that level of 20 mg-N/kg·hr until day 66. At this point, the activity increased steadily to a maximum of 60 mg-N/kg·hr on day 90. This increasing level of activity is likely caused by the increased surface area that adding the extra biobeads on day 45 provided to colonizing denitrifiers in the process. The lag in increasing activity is due to the time taken for the colonizing denitrifiers to grow and populate the new beads. At the same time, the increased biobead load also leads to less turbulence in the reactor, which means the biobeads are not contacting each other with the same energy as when the biobead load is only 10% w/v. This results in more stable growth conditions for surface colonizing biofilms.

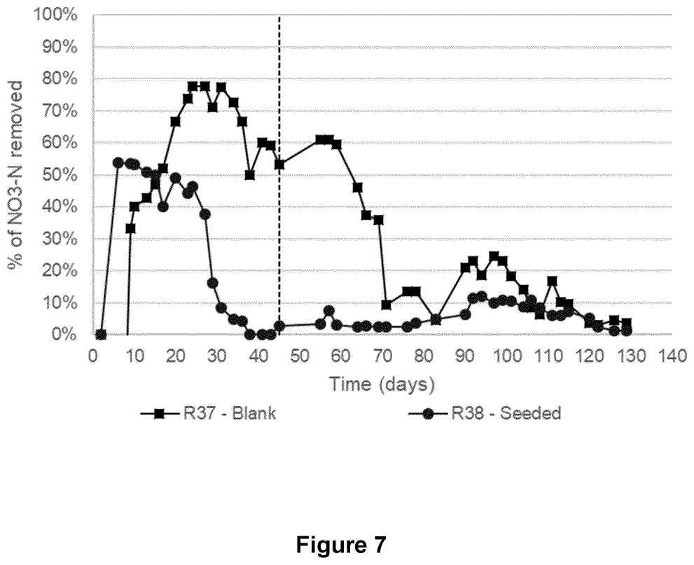

While nitrate removal is the primary objective of drinking water treatment processes, this must be achieved with minimal production of nitrite (NO₂) during the process. While the MCL for nitrate is 10 mg-N/L, it is only 1 mg-N/L for nitrite. Therefore, the formation of nitrite as a by-product in the nitrate removal process was monitored in both reactors throughout the trial. The proportion of the NO₃ removed that was converted to nitrite and left the reactor in the effluent is illustrated in FIG. 7. The results indicate that initially in both reactors the denitrification process often stalled at nitrite. This is likely due to the initial loading of nitrate the biobeads were exposed to in both reactors overwhelmed the microbial mass that was present. This is illustrated clearly as the relative production of nitrite from nitrate was significantly higher in the blank biobeads (R37) where the denitrifying biomass was much lower than in the seeded biobeads (R38). The seeded biobeads were also able to recover much more quickly than the blank biobeads, achieving less than 10% incomplete denitrification after only 31 days, while the blank biobeads were first able to achieve this after 70 days. The benefits of seeding the biobeads with specific denitrifying biomass is clearly demonstrated by these results, especially when looking towards drinking water nitrate removal applications.

### Example 6—Biomass Production

In both reactors, biomass production was evidenced and measured. The results are presented in Table 4. The results indicate that the biobeads do not irreversibly withhold the biomass that is immobilized within the beads. The seeded biobeads in R38 had a TSS production that was on average 50% higher than the blank biobeads in R37. This indicates that the presence of a significantly amount of biomass that has a high activity will lead to increased biomass production. However, the biomass production is significantly lower than what would be expected in a comparable activated sludge process, indicating that the immobilization of the denitrifying microbes in the biobeads may lead to a lower specific biomass yield compared to competing biological denitrification processes.

TABLE 4
Average TSS concentrations in the reactors over
the 129-day period of continuous operation

| | Average | TSS St Dev. |
|---|---|---|
| Reactor | (mg/L) | (mg/L) |
| R37—Blank | 4 | 3 |
| R38—Seeded | 6 | 4 |

### Example 7—Fluorescent In-Situ Hybridization (FISH) Analysis

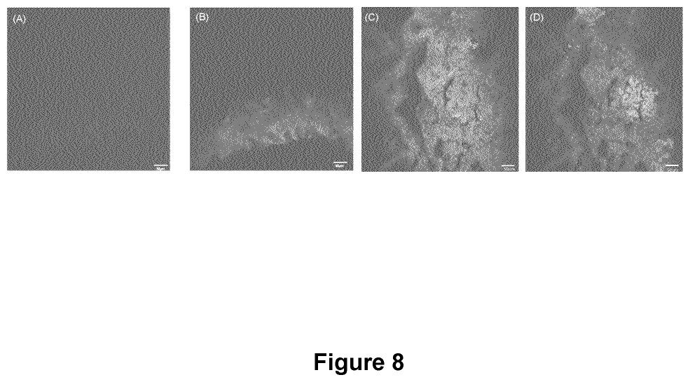

The biobeads in both reactors were sampled for fluorescent in-situ hybridization (FISH) analysis to identify whether the microbes originally seeded in the biobeads were still present and dominant and whether other microbes had been able to colonize the biobeads during operation. The results of this FISH analysis at day 122 are shown in FIG. 8. Pictures A and B are confocal images at 200× amplification and illustrate the relative abundance in the blank R37 biobeads of all Eubacteria (probe EUBmix_Alexa350—cyan) and Paracoccus (probe Par651: Laexa488—green), respectively. Pictures C and D are confocal images at 200× amplification and illustrate the relative abundance of all Eubacteria and Paracoccus, respectively, in seeded biobeads in R38.

Based on this analysis, a qualitative assessment of the presence of Paracoccus pantotrophus was performed on the beads in reactors R37 and R38 on day 44 and day 122, and the results are presented in Table 5.

TABLE 5
Qualitative FISH results indicating the dominance of
Paracoccus pantotrophus in the biobeads from R37 and
R38 after 44 days and 122 days of continuous operation

| | R37-Blank | R38-seeded |
|---|---|---|
| Day 44-Paracoccus | x | xxx |
| pantotrophus | | |
| Day 122-Paracoccus | x | xxxx |
| pantotrophus | | |

x = 0-10% of visible bacteria; xx = 11-20% of visible bacteria; xxx = 20-50% of visible bacteria; xxxx = >50% of visible bacteria

The results of the FISH analysis clearly indicate that despite the biobeads being colonized by indigenous microbes, in the biobeads seeded with Paracoccus pantotrophus the seeded microbes remain the dominant species even after a significant period of operation. In fact, the dominance of the seeded microbe species increases over time. It is expected that this pattern would continue in the longer term as the dominance of the Paracoccus pantotrophus in the biobeads ensures that other microbes can only colonize the surface of the biobeads and thereby preventing them from out-competing the seeded microbes.

### Example 8—Demonstration of Improved Carbon Source Utilization in a TN Removal Biobead Reactor Through the Application of Unique Biotechnology

### BACKGROUND

The removal of total nitrogen (TN) is often required for drinking water and wastewater treatment processes. This can be achieved through a biological process utilizing denitrifying microbes. The process of denitrification requires the oxidation of a carbon source to act as an electron donor to drive the conversion of nitrate (NO₃) and nitrate (NO₂) to nitrogen gas (N₂). Common carbon sources used for denitrification are organic material in wastewater, and external carbon sources such as methanol, ethanol, glycol, molasses and other easily bio-degradable substances. The integration of a denitrifying microbe that can consume less carbon per unit of TN removed represents a significant commercial opportunity as it could significantly reduce the operational expenses of a biological TN removal system.

### Experimental Aims:

Demonstrate the ability to immobilize a carbon source efficient denitrifier (Pseudomonas lini) into a PVA-GA biobead while maintaining low carbon source consumption denitrification capability
Compare the denitrification capabilities of the carbon source efficient denitrifier with a more traditional denitrifying microbe (Paracoccus pantotrophus)

### Method:

### Biobead Preparation

The biobeads are prepared as described in Example 4
   Pseudomonas lini biobeads
   Paracoccus pantotrophus biobeads

### Lab Scale Reactor Operation

Two reactors were operated under identical conditions for a period of 25 days
In R38, biobeads seeded with Paracoccus pantotrophus were used.
In R45, biobeads seeded with were Pseudomonas lini used
General operational conditions at the start of the trials are outlined in Table 6
Tap water was used in the experiments
Nitrate was supplied as NaNO₃
BOD was supplied as a 50:50 (on a BOD basis) mixture of sodium acetate and acetic acid
P was supplied as KH₂PO₄
pH was adjusted as required by addition of 10% HCl
A trace elements solution was applied at a concentration of 60 mg/L

TABLE 6
Operational conditions for the reactors from Day 1-25

| Biobead | Reactor conditions | | | | | Substrate characteristics | | |
|---|---|---|---|---|---|---|---|---|
| loading | Volume | Temperature | | HRT | DO | NO₃ | BOD | P |
| (% w/v) | (L) | (deg C) | ph | (h) | (mg/L) | (mg-N/L) | (mg/L) | (mg-P/L) |
| 10 | 0.5 | 15 | 7-8 | 0.5 | 0-1 | 10 | 50 | 2.5 |

### Analytical Methods

Sampling was undertaken three (3) times per week by a certified analytical laboratory
Standard methods known to one skilled in the art were used to measure the components of the feed water and the water coming from the reactors listed in Table 1

### Results and Discussion:

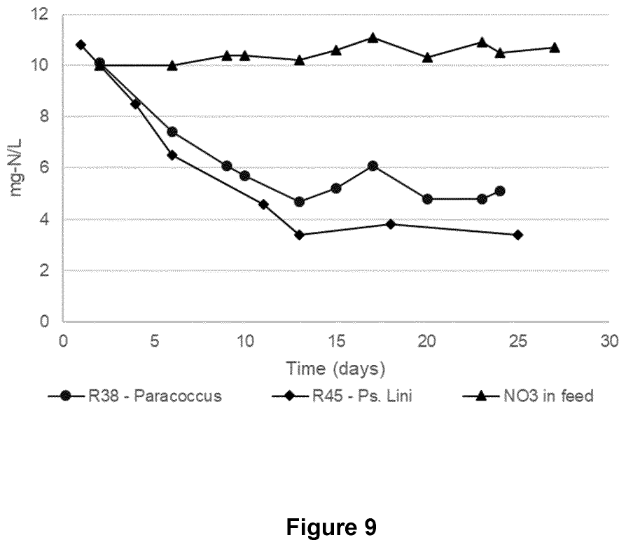

The reactors were started up using identical operational conditions as outlined in Table 6. The concentration of total nitrogen (TN) was monitored in the influent and effluent of the reactors (FIG. 9). Initially, the two reactors had an effluent TN concentration that was decreasing at a similar rate over time. Effluent TN concentration of the biobeads in R38 were lowered by a dramatic degree, with a 50% reduction within 10 days. However, even more remarkably, from day 6 onwards, the biobeads in R45 were able to achieve a lower effluent TN concentration compared to the biobeads in R38.

This performance was also reflected in the specific activity of the biobeads (mg-N removed per kg of biobeads per hr (mg-N/kg·hr)) as illustrated in FIG. 10. The biobeads in R45 demonstrated from an early stage a significantly higher rate of TN removal, eventually achieving a rate that was approximately 50% higher than the biobeads in R38.

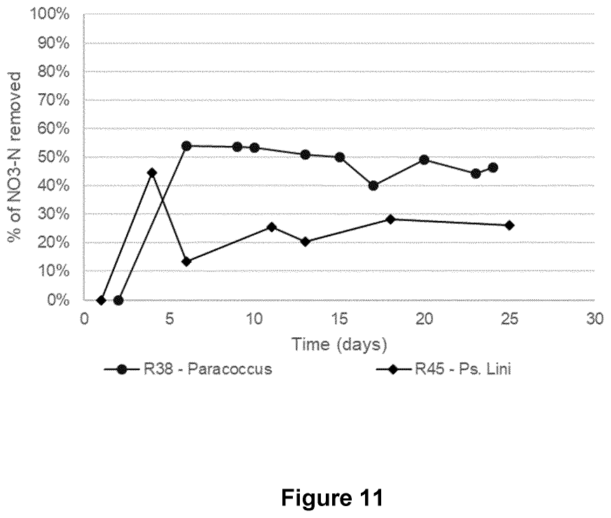

The significantly higher TN removal activity evidenced in R45 was due to the higher conversion of nitrate to nitrogen gas (complete denitrification) by the biobeads seeded with Pseudomonas lini (R45) compared to those seeded with Paracoccus pantotrophus (R38). This is illustrated in FIG. 11, where it is clear the biobeads in R45 have a higher complete denitrification capability compared to the biobeads in R38. This is important, as nitrite is a component of TN measurements, and therefore converting nitrate to nitrite is not useful for meeting TN treatment objectives. This result further illustrates that the biobeads in R45 have a denitrification microbe that is significantly more efficient at utilizing carbon sources for denitrification than the denitrifiers integrated into the biobeads in R38.

It would be expected that the lower rates of TN removal and lower conversion of nitrate to nitrogen gas would mean the carbon source consumption in R38 would be significantly lower than that in R45. However, surprisingly, this was not the case. In Table 7, the average consumption of COD per unit of TN removed is shown. The results show that the biobeads with the Pseudomonas lini (R45) were on average 28% more efficient at removing TN compared to the biobeads with Paracoccus pantotrophus (R38). This represents a potential saving of 28% on external carbon source required by a TN treatment plant to achieve denitrification, which has significant commercial advantages for the biobead system operator.

TABLE 7
Average consumption of COD per unit of TN removed
in each of the reactors R38 and R45 over a period
of 25 days of continuous operation

| | Average COD consumed | |
|---|---|---|
| | per unit TN removed | Standard Deviation |
| | (mg-COD/mg-TN) | (mg-COD/mg-TN) |
| R38- | 4.86 | 3.06 |
| R45- | 3.48 | 1.51 |

### Example 9 Demonstration of the Efficacy of Biobeads for Total Ammonia Nitrogen (TAN) Treatment from Wastewater

### Background

Ammonia is a highly problematic pollutant for several reasons—it is toxic to aquatic wildlife and it can lead to excessive growth of algae in receiving waters leading to oxygen depletion or eutrophication. Therefore, one of the key objectives of wastewater treatment is the removal of ammonia from the wastewater. This is generally done using biological wastewater treatment methods, where nitrifying bacteria are exploited to convert ammonia to nitrite and then nitrate. Nitrification bacteria are generally recognized as being sensitive to process changes and relatively slow growing compared to other bacteria in the wastewater treatment process. For example, in an activated sludge type nitrification process at a municipal wastewater treatment plant, despite the operation of the system to encourage nitrification (long solids retention time), the total biomass that could be classified as nitrifiers is generally less than 10%. Therefore, nitrification processes can be relatively easily impacted negatively by toxins, changing process conditions or poor operation leading to a loss of nitrification capacity. Given the relatively slow growth rate of nitrifiers, the recovery time after such an upset may be over a period of weeks rather than days. With this knowledge, it is clear that there would be clear advantages to developing a nitrification process that utilized immobilized or encapsulated nitrifiers (biobeads). In such a system, the only biomass in the process would be nitrifiers as this is what is being added to biobeads, allowing for a significant increase in the concentration of nitrifiers in the treatment process and increasing the oxygen utilization efficiency of the process as only nitrifiers would be consuming dissolved oxygen. The biobeads also provide an environment where the nitrifiers are protected from process changes, toxins and other shocks that would normally have a strong negative impact on the nitrification capacity of the treatment process.

### Experimental Aims:

Demonstrate the efficacy of biobeads seeded with Prawnbac, (Novozymes AS) in a system treating a wastewater with a concentration of ammonia that represents a very high concentration for municipal wastewater treatment (60 mg-N/L)

### Method:

Biobead preparation. The biobeads are prepared as described in Example 4
Blank biobeads
Prawnbac biobeads
   Lab scale reactor operation
Two reactors were operated under identical conditions for a period of 21 days
In M06, blank biobeads were used. In M07, biobeads seeded with Prawnbac (Novozymes AS) nitrification product were used
General operational conditions at the start of the trials are outlined in Table 8
Tap water was used in the experiments
Ammonia was supplied as NH4Cl
P was supplied as KH2PO4
Alkalinity was supplied in a ratio of 10:1 compared to NH4-N. Alkalinity was supplied as CaCO3
pH was adjusted as required by addition of 10% HCl or 10% NaOH
A trace elements solution was applied at a concentration of 60 mg/L

TABLE 8
Operational conditions for the reactors from Day 1-21

| Biobead | Reactor conditions | | | | | Substrate characteristics | | |
|---|---|---|---|---|---|---|---|---|
| loading | Volume | Temperature | | HRT | DO | NO₄ | Alkalinity | P |
| (% w/v) | (L) | (deg C) | ph | (h) | (mg/L) | (mg-N/L) | (mg-CaCOY/L) | (mg-P/L) |
| 10 | 1.0 | 10 | 7.5-8.5 | 3.0 | >4 | 50 | 500 | 2.5 |

### Analytical Methods

Sampling was undertaken three (3) times per week by a certified analytical laboratory

### Results and Discussion:

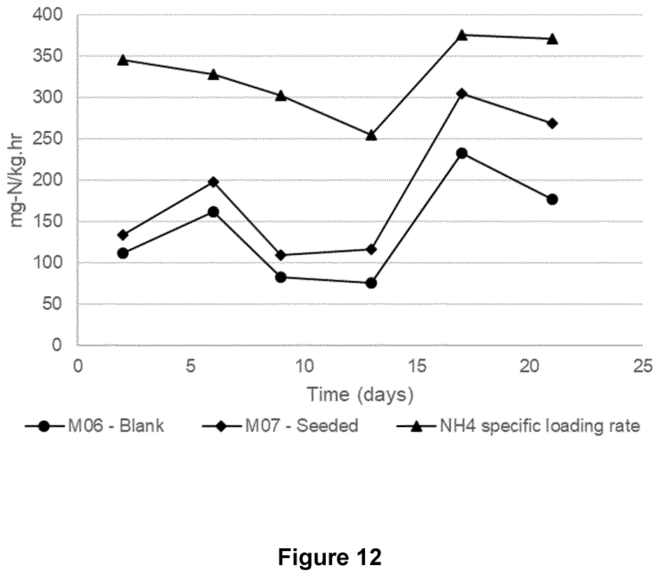

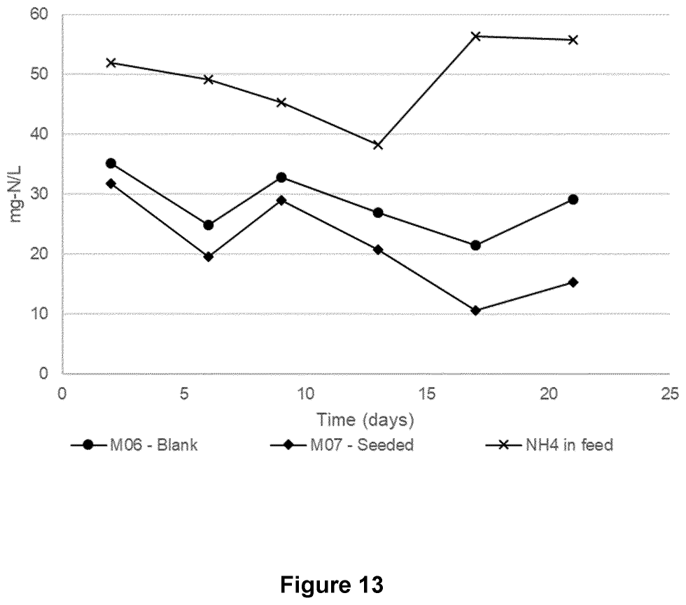

The reactors were started up using identical conditions. There was a significant lag period for both reactors with relatively similar and stable levels of specific ammonia treatment rates (mg-N/kg·hr) (FIG. 12) and ammonia effluent concentration (mg-N/L) (FIG. 13). From day 9, there was a divergence in performance, with the seeded biobead reactor M07 exhibiting increasingly high specific ammonia treatment rates and a decreasing ammonia concentration in the effluent compared to the blank biobeads in reactor M06. After 21 days of continuous operation, the seeded biobeads in M07 delivered 40% higher specific ammonia treatment rate compared to the blank beads in M06. Similarly, the seeded biobeads were removing 73% of the ammonia fed to the reactor, while the blank biobeads were only treating 48% of the influent ammonia.

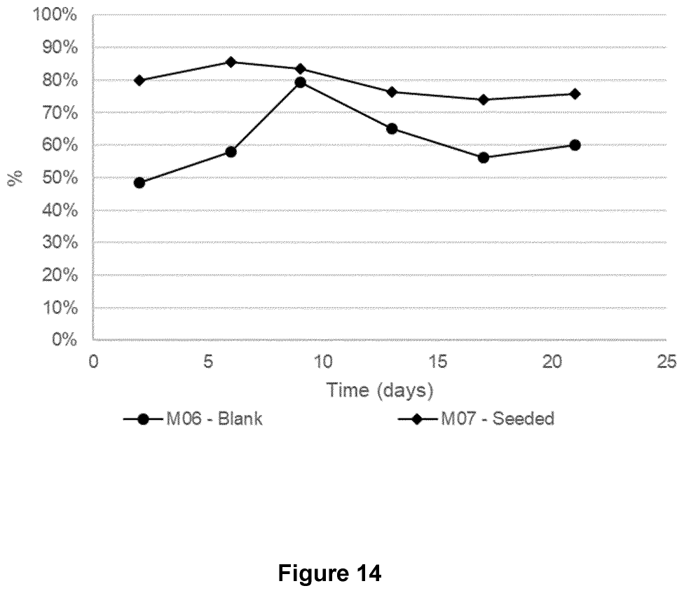

While the performance of the two reactors was similar with regards to ammonia treatment, it was clear from the start of the trials that the seeded biobeads had a significantly greater capacity to complete the nitrification process (convert ammonia to nitrate) as illustrated in FIG. 14. After 9 days of operation, the difference between the nitrification capacity of the seeded and blank biobeads was reduced. This is likely due to the ability of the blank beads to build up a more balanced colonizing community of nitrifiers that were able to more effectively convert ammonia to nitrate. However, at no time during the trial were the blank biobeads able to demonstrate an ability to achieve full nitrification of the ammonia as effectively as the seeded biobeads. This clearly demonstrated the value of utilizing seeded biobeads compared to blank biobeads or carriers to achieve an efficient nitrification process.

### Example 10 Demonstration of the Positive Effect Microbe Encapsulation has on Protecting Microbes from the Effects of Exposure to Free Chlorine

A key advantage of the invention of encapsulating microbes in the polymeric biobead is the that encapsulation in a polymeric bead provides an increased level of protection to the microbes within from potentially toxic components in the water being treated when compared to microbes in suspended or fixed film growth in the water. Biological nitrogen removal processes are often exposed to toxins or inhibitors as the influent of water treatment systems is not controlled (for example municipal sewer) or are produced as part of an upstream production process in industrial wastewater treatment plants. One such toxin that can have a significant impact on biological nitrogen removal processes is chlorine, which will kill microbes on contact. Therefore, testing the effect of free chlorine on the activity of denitrifying biobeads and their ability to recover after exposure to chlorine, will provide a suitable model for demonstrating the efficacy of biobeads at protecting the microbes housed within.

### Experimental Aims:

Demonstrate the efficacy of biobeads to protect the microbes within from prolonged exposure to 5 mg/L of free chlorine
Demonstrate that biobeads seeded with the denitrifying micro-organism Paracoccus pantotrophus can recover activity to pre-exposure levels within a short period of time
Compare the performance of biobeads seeded with microbes to blank biobeads where denitrification activity comes only from microbes on the surface of the biobeads

### Methods:

Biobead preparation
   The biobeads are prepared as described in Example 4
      Blank biobeads
      Seeded biobeads
Lab scale reactor operation
   Two reactors were operated under identical conditions for a period of 129 days
   In R37, blank biobeads were used. In R38, seeded biobeads were used
   General operational conditions during trials are outlined in Table 9
      Tap water was used in the experiments
      Nitrate was supplied as NaNO₃
      BOD was supplied as a 50:50 (on a BOD basis) mixture of sodium acetate and acetic acid
      P was supplied as KH2PO4
      pH was adjusted as required by addition of 10% HCl
      A trace elements solution was applied at a concentration of 60 mg/L

TABLE 9
Operational conditions for the reactors from Day 1-21

| Biobead | Reactor conditions | | | | | Substrate characteristics | | |
|---|---|---|---|---|---|---|---|---|
| loading | Volume | Temperature | | HRT | DO | NO₃ | BOD | P |
| (% w/v) | (L) | (deg C) | ph | (h) | (mg/L) | (mg-N/L) | (mg/L) | (mg-P/L) |
| 20 | 0.5 | 15 | 7-8 | 0.5 | 0-1 | 10 | 50 | 2.5 |

Chlorine was added as part of the reactor feed from day 14-17 as sodium hypochlorite at a concentration of 5 mg/L of chlorine
Analytical methods
   Sampling was undertaken two (2) to three (3) times per week by a certified analytical laboratory
   Standard methods known to one skilled in the art were used to measure the components of the feed water and the water coming from the reactors listed in Table 2

### Results and Discussion:

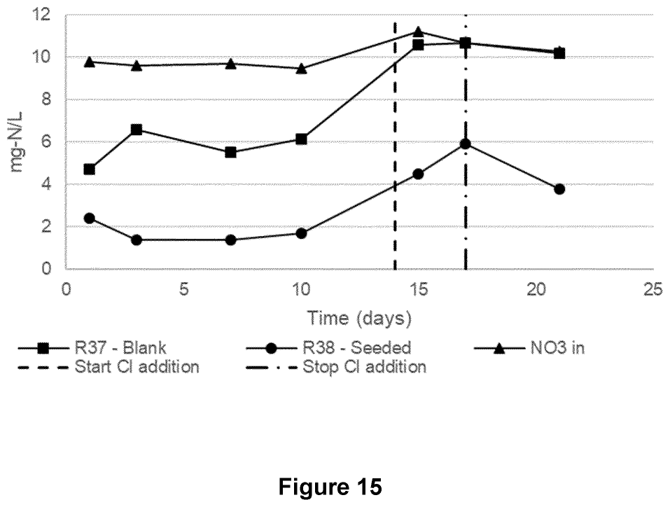

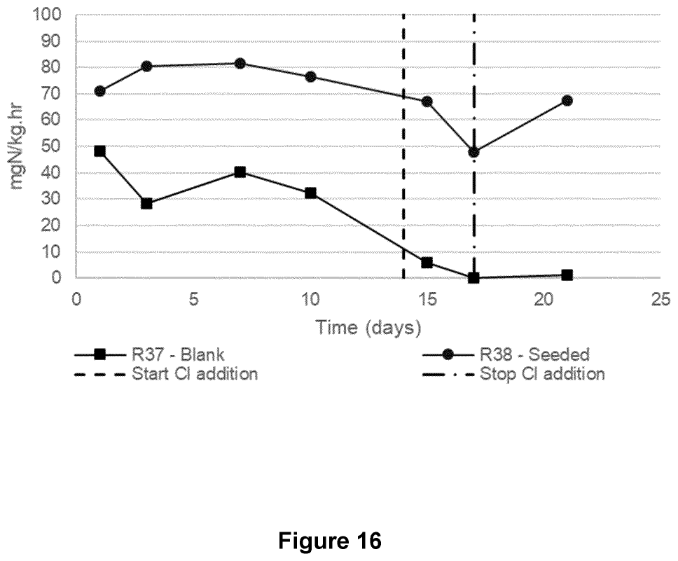

The concentration of nitrate and nitrite in the influent (feed) and effluent from the reactors was measured frequently throughout the trial period. FIG. 15 illustrates the concentration of total nitrogen (nitrate+nitrite) in both the influent and effluent of both R37 and R38. FIG. 16 demonstrates the specific total nitrogen (TN) removal rate for the biobeads in both R37 and R38. The dotted lines at day 14 indicates where the sodium hypochlorite started being added to the reactor feed, with the second dashed lines at day 17 indicating where the addition of sodium hypochlorite stopped. Biobeads in both reactors were negatively impacted by the addition of the chlorine, with increased levels of total nitrogen (TN) in the effluent of both reactors (FIG. 15). After one day of exposure to the chlorine, the blank beads in R37 lost all denitrification activity, with this activity not recovering even after stopping the exposure to chlorine at day 17. In R38, the seeded beads only lost approximately 12% of their TN removal activity after one day of exposure, and 37% of activity after three days of exposure. After the addition of chlorine to the reactors was stopped at day 17, the blank biobeads in R37 did not recover any activity after 4 days. However, the seeded biobeads in R38 recovered a significant proportion of the activity lost during the exposure to chlorine, with and expectation that a full recovery to pre-exposure levels of activity would occur within seven days (FIG. 16).

The exposure to the chlorine did not impact the physical stability of the biobeads (blank or seeded) in any way.

### CONCLUSIONS

The experiment presented clearly demonstrates that the biobeads are able to provide a protective environment for microbes that have been integrated as part of the production process of the biobeads. While microbes are able to colonize the outer surfaces of blank (or not seeded) biobeads and provide denitrification activity, these microbes are not protected against exposure to toxins or inhibitors like those that are integrated into the biobeads.

Biological nitrogen removal processes are often exposed to toxins and inhibitors, with chlorine being one of the most effective microbial contamination control chemicals used, with chlorine used to ensure the disinfection of drinking water by killing any suspended microbes in the water. This experiment has demonstrated that a biological nitrogen removal process utilizing biobeads with integrated microbes would be resistant to the impact of chlorine (or other toxin) exposure and would recover its nitrogen removal activity in a very short period of time. This would have significant value for any operator using a biobead system as the biobeads would ensure toxic exposure would have a minimal impact on TN removal compared to a comparable suspended or fixed film system.

1. A polymer support comprising microorganisms immobilized within a polymer hydrogel, said polymer hydrogel comprising:
polyvinyl alcohol polymeric chains cross linked with glutaraldehyde; and
water or an aqueous solution.

2. The polymer support of claim 1 wherein the polyvinyl alcohol polymeric chains are crosslinked by covalent bonds with the glutaaraldehyde.

3. The polymer support of claim 1 wherein the polyvinyl alcohol polymeric chains consist of 500-3000 monomer units and have a molecular weight of 22.000 g/mol to 130.000 g/mol.

4. A polymer support of claim 1, wherein the immobilized microorganisms comprise ammonium oxidizing microorganisms, nitrite oxidizing microorganisms, and/or denitrifying microorganisms.

5. The polymer support of claim 1, wherein the immobilized microorganisms comprise a combination of ammonium oxidizing microorganisms and nitrite oxidizing microorganisms.

6. The polymer support of claim 1, wherein the immobilized microorganisms Nitrosomonas spp., Nitrobacter spp., Nitrosococcus spp., Nitrosospira spp., Nitrosolobus spp., and Nitrosovibrio spp., Nitrotoga spp., Nitrospira spp. Pseudomonas spp., Paracoccus spp., Hyphomicrobium spp., Castellaniella spp., Janthinobacterium spp., Acidovorax spp., Aeromonas spp., Cellulomonas spp., Buttiauxella spp., Microvirgula spp., Klebsiella spp., Shewanella spp., Pelosinus spp., Variovorax spp., Hydrogenophaga spp., Raoultella spp., Bacillus spp., Achromobacter spp., Ochrobactrum spp., Flavobacterium spp., and/or Delftia.

7. The polymer support of claim 1, wherein the immobilized microorganisms comprise Paracoccus pantotrophus, Paracoccus versutus, Paracoccus denitrificans, Castellaniella defragrans, Pseudomonas proteolytica, Pseudomonas alcaliphila, Pseudomonas chlororaphis, Pseudomonas monteilii, Pseudomonas lini, Pseudomonas alkylphenolica, Pseudomonas nitroreducens, Pseudomonas stutzeri, Ochrobactrum anthropic, Ochrobactrum intermedium, Aeromonas media, Aeromonas veronii, Aeromonas jandaei, Aeromonas hydrophila, Cellulomonas chitinilytica, Cellulomonas cellasea, Cellulomonas hominis, Flavimobis soli, Flavobacterium banpakuense, Buttiauxella agrestis, Buttiauxella noackiae, Achromobacter denitrificans, Pelosinus fermentans, Variovorax dokdonensis, Hydrogenophaga bisanensis, Raoultella terrigena, Delftia lacustris, Shewanella putrefaciens, Acidovorax soli, Hyphomicrobium denitrificans, Nitrosomonas eutropha, Nitrosomonas europaea, Nitrobacter winogradsky, Microvirgula aerodenitrificans, Candidatus kuenenia, Candidatus brocadia, Candidatus anammoxoglobus, Candidatus jettenia, and/or Candidatus scalindua.

8. The polymer support of claim 1, wherein the immobilized microorganisms comprise Pseudomonas lini, Paracoccus pantotrophus, Paracoccus pandtodrophus, Castellaniella defragans, Pseudomonas proteolytica, Paracoccus versutus, Paracoccus denitrificans, Nitrosomonas eutropha, Nitrosomonas europaea, and/or Nitrobacter winogradsky,

9. The polymer support of claim 1, wherein the immobilized microorganisms comprise Nitrosomonas eutropha Nitrosomonas europaea and/or Nitrobacter winogradskyi.

10. The polymer support of claim 1, wherein the immobilized microorganisms comprise Pseudomonas lini, Paracoccus pantotrophus, and/or Paracoccus versutus.

11. The polymer support of claim 1, wherein the immobilized microorganisms comprise Pseudomonas lini.

12. The polymer support of claim 1, wherein the immobilized microorganisms comprise Paracoccus pantotrophus.

13. The polymer support of claim 1, wherein the immobilized microorganisms comprise Castellaniella defragans.

14. The polymer support of claim 1 wherein the immobilized microorganisms comprise Nitrosomonas eutropha.

15. The polymer support of claim 1, wherein the immobilized microorganisms comprise Nitrosomonas europaea.

16. The polymer support of claim 1, wherein the immobilized microorganisms comprise Nitrobacter winogradsky.

17. The polymer support of claim 1, wherein the polymer support is porous.

18. (canceled)

19. The polymer support of claim 1, wherein at least a portion of the polymer support comprises micropores with an average pore diameter of 5 to 40 microns.

20. The polymer support of claim 1, wherein at least a portion of the polymer support comprises micropores with an average pore diameter of 10 to 40 microns.

21. The polymer support of claim 1, comprising a microbial load of 5 g/kg to 250 g/kg, based upon the total weight of the polymer support.

22-30. (canceled)
